(19) United States
(12) Patent Application Publication
(10) Pub. No.: US 2012/0213818 A1
(43) Pub. Date: Aug. 23, 2012

(54) COMBINATION THERAPY FOR IMMUNOSTIMULATION

(75) Inventors: Ingmar Hoerr, Tuebingen (DE); Steve Pascolo, Tuebingen (DE)

(73) Assignee: CUREVAC GmbH

(21) Appl. No.: 13/361,686

(22) Filed: Jan. 30, 2012

Related U.S. Application Data
(63) Continuation of application No. 10/580,746, filed on Sep. 29, 2006, now abandoned.

(30) Foreign Application Priority Data
Sep. 2, 2004 (DE) 10 2004 042 546.9

(51) Int. Cl. A61K 39/00; A61K 39/39; A61K 39/02; A61P 31/04; A61P 37/08; A61P 37/06; A61P 31/12; A61K 39/145; A61P 35/00

(52) U.S. Cl. 424/209.1; 424/184.1; 424/277.1; 424/234.1; 424/204.1

(57) ABSTRACT

The present invention relates to a method for immunostimulation in a mammal which comprises

a. administration of at least one mRNA containing a region which codes for at least one antigen of a pathogen or at least one tumour antigen, and b. administration of at least one cytokine, at least one cytokine mRNA, at least one CpG DNA or at least one adjuvant RNA. The invention likewise relates to a product and a kit comprising the mRNA and cytokine or cytokine mRNA or CpG DNA or adjuvant RNA of the invention.

The present invention relates to a method for immunostimulation in a mammal, wherein the method comprises administration of an mRNA which codes for an antigen of a pathogenic microorganism, and administration of at least one cytokine, in particular GM-CSF, at least one cytokine mRNA, at least one CpG DNA, at least one adjuvo-viral mRNA and/or at least one adjuvant RNA.

Satisfactory results in connection with numerous diseases can be achieved with conventional vaccines which comprise attenuated or inactivated pathogens and further substances, such as sugars or protein contents. However, it is not possible to achieve an adequate protection against a large number of infectious organisms, such as, for example, HIV or Plasmodium falciparum, and in particular against tumours with such vaccines. There is moreover the risk that new pathogens arise doe to undesirable recombination events (such as e.g. in the case of the BARS epidemic).

Methods of molecular medicine, such as gene therapy and genetic vaccination, therefore play a large role in the therapy and prevention of numerous diseases. These methods are based on the introduction of nucleic acids into cells or tissue of the patient, followed by processing of the information coded by the nucleic acids introduced, i.e. expression of the desired polypeptides or proteins. Both DNA and RNA are possible as nucleic acids to be introduced.

Genetic vaccinations, which consist of injection of naked plasmid DNA, were demonstrated on mice for the first time in the early 90s. However, it emerged in clinical phase I/II trials that in humans this technology was not able to fulfil the expectations aroused by the studies on mice (6). Numerous DNA-based genetic vaccinations have since been developed. Various methods for introducing DNA into cells have been developed in this connection, such as e.g. calcium phosphate transfection, polyprene transfection, protoplast fusion, electroporation, microinjection and lipofection, lipofection in particular having emerged as a suitable method. The use of DNA viruses as the DNA vehicle is likewise possible. Because of their infection properties, such viruses have a very high transfection rate. The viruses used are genetically modified in this method, so that no functional infectious particles are formed in the transfected cell. In spite of this safety precaution, however, a risk of uncontrolled propagation of the genetherapeutically active genes introduced and she viral genes introduced cannot be ruled out e.g. because of possible recombination events, in addition, DNA vaccination has further potential safety risks (7, 8). The recombinant DNA injected must first teach the cell nucleus, and this step can already reduce the efficiency of DNA vaccination. In the cell nucleus, there is the danger that the DNA integrates into the host genome. Integration of foreign DNA into the host genome can have an influence on expression of the host genes and possibly trigger expression of an oncogene or destruction of a tumour suppressor gene. A gene—and therefore the gene product—which is essential to the host may likewise be inactivated by integration of the foreign. DNA into the coding region of this gene. There is a particular danger if integration of the DNA takes place into a gene which is involved in regulation of cell growth. In this case, the host cell may enter into a degenerated state and lead to cancer or tumour formation.

Moreover, for expression of a DNA introduced into the cell, it is necessary for the corresponding DNA vehicles to contain a potent promoter, such as the viral CMV promoter. Integration of such promoters into the genome of the treated cell can lead, to undesirable changes in the regulation of gene expression in the cell. A further disadvantage is that the DNA molecules remain in the cell nucleus for a long time, either as an episome or, as mentioned, integrated into the host genome. This leads to a production of the transgenic protein which is not limited or cannot be limited in time and to the danger of an associated tolerance towards this transgenic protein. The development of anti-DNA antibodies (9) and the induction of autoimmune diseases can furthermore be triggered by injection of DNA.

All these risks listed which are associated with genetic vaccination do not exist if messenger RNA (mRNA) is used instead of DNA. For example. mRNA does not integrate into the host genome, if RNA is used as a vaccine, no viral sequences, such as promoters etc., are necessary for effective transcription etc. RNA is indeed far more unstable than DNA (RNA-degrading enzymes, so-called RNases (ribonucleases), in particular, but also numerous further processes which destabilise RNA are responsible for the instability of RNA), but methods for stabilising RNA have meanwhile been disclosed in the prior art. Thus, for example, in WO 03/051401, WO 02/098443, WO 99/14346, EP-A-1083232, U.S. Pat. No. 5,580,859 and U.S. Pat. No. 6,214,804. Methods have also been developed for protecting RNA against degradation by ribonucleases, which are carried out using liposomes (15) or an intra-cytosolic in vivo administration of the nucleic acid with a ballistic device (gene gun) (16). An ex vivo method which relates to transfection of dendritic cells has likewise been presented (12).

For an RNA-based vaccination, inter alia, immunisation strategies which are based on self-replicating RNA which code both for an antigen and for a viral RNA replicase have been developed (13, 14). Such methods are indeed efficient, but there are safety risks in the use of viral RNA replicases in genetic vaccines (recombination between the RNA injected and the endogenous RNA could lead to the formation of new types of alpha viruses).

Overall, it is to be said that no mRNA vaccine which ensures triggering of an immune response in the organism to which it is administered, increases this response and at the same time largely avoids undesirable side effects is described in the prior art.

A further great disadvantage of the snRNA vaccines known in the prior art is that only a humoral immune response (Th2 type) is triggered by an siRNA vaccination. However, all viruses and numerous bacteria, such as, for example, mycobacteria and parasites, penetrate into the cells, multiply/proliferate there and are thus protected from antibodies. In order therefore to cause an antitumoral or antiviral immune response in particular, it is necessary to trigger a cellular immune response (Th1 type).

The object of the present invention is accordingly to provide a novel system for gene therapy and genetic vaccination which, ensures a more effective immune response and therefore a more effective protection, in particular against intracellular pathogens and the diseases caused by these pathogens, or also against tumours.

This object is achieved by the embodiments of she present invention characterized in the claims.

The present invention provides a method for immunostimulation in a mammal, comprising the following steps:
a. administration, of at least one mRNA containing a region which codes for at least one antigen of a pathogen or at least one tumour antigen and
b. administration of at least one component, chosen from, the group consisting of at least one cytokine, at least one cytokine mRNA, at least one CpG DNA, at least one adjuvo-viral mRNA and at least one adjuvant RNA.

In the following, the mRNA which codes for at least one antigen from a pathogen or at least one tumour antigen is called “mRNA according to the invention”. This is the mRNA employed in step (a.) of the method according to the invention. This can be in a modified or non-modified form.

The invention is based on the finding that injection of naked stabilized mRNA causes a specific immune response (17). Such an antigen-specific immune response has been investigated in more detail according to the invention, in particular in comparison, with a DMA-induced immune response. For this, in one experimental set-up naked stabilised mRNA and in another experimental set-up plasmid DNA was injected into the ear of BALB/c mice. In both experimental set-ups, the nucleic acids contained a region coding for β-galactosidase. It was to be found as the result that in the case of the mRNA vaccination, chiefly IgG1 antibodies were produced, while in the case of the DNA vaccination, chiefly IgG2a antibodies were formed. It was thus possible to demonstrate according to the invention that mRNA vaccination causes a humoral immune response (Th2) (production of IgG1), while DNA vaccination causes a cellular immune response (Th1) (production of IgG2a). Surprisingly, it was also accordingly to be found by this study that the decision as to whether a humoral or cellular immune response is triggered in a mammal, here in mice, depends neither on the administration route nor on the antigen which is coded by the nucleic acid, but rather on the nature of the nucleic acid, RNA or DNA. Nucleic acids which, instead of the region coding β-galactosidase, contained a region which coded for an antigen of a pathogen or a tumour antigen were used in further experimental set-ups. Such an antigen coding regions are discussed in more detail in the following. The results described above in respect of triggering of a Th1 or Th2 immune response were likewise found in these experimental set-ups. The dosage of the mRNA according to the invention depends in particular on the disease to be treated and the stage of progression thereof, and also the body weight, the age and the sex of the patient (the terms organism, mammal, human and patient are used synonymously in the context of the invention). The concentration of the mRNA according to the invention can therefore vary within a range of from approximately 1 μg to 100 mg/ml.

It has moreover been found according to the invention that particularly advantageous properties are established if the mRNA according to the invention is administered in combination with at least one component of at least one of the following categories, namely cytokine, cytokine mRNA, CpG DNA, adjuvo-viral mRNA and/or adjuvant RNA. Components of the abovementioned categories have adjuvant properties, as is found according to the invention, so that the compounds or components falling under these categories are to be regarded as adjuvants. These adjuvant properties are based on the effect of the compounds of the above-mentioned categories of having an immunostimulatory action. Components from the categories of cytokines or cytokine-expressing cytokine mRNAs already have a direct immunostimulatory action as such. Compounds of the other above-mentioned categories can have an indirect immunostimulatory action in that they stimulate cytokine secretion in the organism treated (human or animal, in particular domestic pets).

The inventors have accordingly investigated the influence of cytokines on RNA vaccination. Cytokines represent an outstanding adjuvant in connection with DNA vaccinations—as is known from the prior art (19, 20, 24, 25). A preferred cytokine is GM-CSF (granulocyte macrophage colony stimulating factor), which increases the density of dendritic cells (DCs) in the skin and thus intensifies an immune response caused by a DNA vaccination. The aim of the investigations according to the invention was also to intensity still further, by administration of cytokines, an mRNA-induced immune response according to the invention. The administration of cytokines in combination with peptides (26) and DNA (27) is known in the prior art. Nevertheless, on the one hand it has not hitherto been possible to achieve satisfactory results, probably (also) because it has not been possible to specify a suitable point in time for administration of GM-CSF, and on the other hand vaccinations carried out with peptides or DNA cannot be applied to RNA-based vaccinations. This has already been discussed in detail above.

According to the invention, parallel experiments were carried out in which the administration of a cytokine in protein form, preferably administration of GM-CSF, was carried out at various points in time before, after and simultaneously with an mRNA vaccination (the mRNA (according to the invention) coding for β-galactosidase, an antigen of a pathogen or a tumour antigen). It was to be found as the result that an administration before the vaccination exerted no substantial effect on the quality or quantify (type and amount of the immunoglobulin IgG1/IgG2a produced) (see FIG. 3 for β-galactosidase). Surprisingly, however, it was to be found according to the invention that if a cytokine, preferably GM-CSF, is administered after the mRNA vaccination, not only was there an increased Th2 immune response, but moreover a Th1 immune response was also induced (see FIG. 3 and Table 1). Particularly good results were obtained if a cytokine, preferably GM-CSF, was administered preferably approximately 24 hours after administration of the mRNA according to the invention.

Moreover, corresponding experiments were also carried out in which, instead of the cytokine in protein form, the administration of a cytokine mRNA (i.e. an mRNA which contains the coding region for a functional cytokine, a fragment or a variant thereof), preferably a G-CSF, M-CSF or GM-CSF mRNA administration, was carried out at various points in time before, after and simultaneously with an mRNA vaccination (the mRNA (according to the invention) coding for β-galactosidase). The result of the administration, expressed by the secretion of a cytokine (IFN-γ) can be seen from FIG. 5, Surprisingly, according to the invention it was also to be found here that if cytokine mRNA, preferably GM-CSF mRNA, is administered before, simultaneously with and after the mRNA vaccination, a great increase in IFN-γ secretion takes place, as a result of which an indirectly immunostimulatory action is caused. Particularly good results were obtained in particular if cytokine mRNA, preferably GM-CSF mRNA, was administered preferably approximately 24 hours after administration of the mRNA according to the invention.

Corresponding results were achieved on administration of CpG DNA before, after and simultaneously with the mRNA vaccination described above, CpG represents a relatively rare dinucleotide sequence in DNA, in which the cytosine residue is often methylated, so that 5-methylcytosine is present. The methylation of the cytosine residue has effects on gene regulation, such as e.g. inhibition of the binding of transcription factors, blockade of promoter sites etc.). That is to say, here also not only was there an increased Th2 immune response, but moreover a Th1 immune response was induced. Here also, particularly good results were achieved if the GpG DNA was administered approximately 24 hours after administration of the mRNA according to the invention. In particular, CpG DNA with the motif CpG DNA 1668 with the sequence 5′-TCC ATG ACG TTC CTG ATG CT-3′ or the motif CpG 1982 5′-TCP AGG ACT TCT CTC AGG TT-3′ was used in the experiments.

Administration of adjuvo-viral mRNA was also capable of triggering an immunostimulatory effect. In this case, cytokine secretion is likewise brought about. mRNAs which code for the influenza matrix protein or the HES surface protein are be mentioned as examples of such adjuvo-viral mRNAs. Overall, those antigens which represent viral matrix or surface proteins are typically usable for an adjuvant action of an adjuvo-viral mRNA.

Corresponding results were achieved on administration of adjuvant RNA before, after and simultaneously with the mRNA vaccination described above. The adjuvant RNA comprises relatively short RNA molecules which consist e.g. of about 2 to about 1,000 nucleotides, preferably about 8 to about 200 nucleotides, particularly preferably 15 to about 31 nucleotides. According to the invention, the adjuvant RNA can likewise be in single- or double-stranded form. In this context, in particular, double-stranded RNA having a length of 21 nucleotides can also be employed as interference RNA in order to specifically switch off genes, e.g. of tumour cells, and thus to kill these cells in a targeted manner, or in order to inactivate genes active therein which are to be held responsible for a malignant degeneration (Elbashir et. al., Nature 2001, 411, 494-498). The adjuvant RNA is employed in step (b.) in the method according to the invention and is preferably modified chemically, as disclosed in the following in connection with modifications. The adjuvant RNA activates cells of the immune system (chiefly antigen-presenting cells, in particular dendritic cells (DC), and the defense cells, e.g. in the form of T cells) to a particularly high degree and thus stimulates the immune system of an organism. The adjuvant RNA leads here, in particular, to an increased release of immune-controlling cytokines, e.g. interleukins, such as IL-6, IL-12 esc.

The dosage of the cytokine or cytokine mRNA or CpG DNA or adjuvo-viral mRNA or adjuvant RNA depends on she mRNA according to the invention, which is used, which contains a coding region for an antigen from a pathogen or for a tumour-antigen, the disease to be treated, the condition of the patient to be treated (weight, height, progression status of the disease etc.). The dosage range is approximately in a concentration range of from 5 to 300 μg/m².

“vaccination” or “inoculation” in general means the introduction of one or more antigens or, in the context of the invention, the introduction of the genetic information for one or more antigen(s) in the form of the mRNA according to the invention which codes for the antigen(s) into an organism, in particular into one/several cell/cells or tissue/tissues of this organism. The mRNA according to the invention administered in this way is translated into the antigen in the organise or in the cells thereof, i.e. the antigen coded by the mRNA according to the invention (also: antigenic polypeptide or antigenic peptide) is expressed, as a result of which an immune response directed against this antigen is stimulated.

An “immunostimulation” or “stimulation of an immune response” as a rule tabes place by infection of a foreign organism (e.g. a mammal, in particular a human) with a pathogen (or also pathogenic organism). In the context of the invention, a “pathogen” or “pathogenic organism” includes, in particular, viruses and bacteria, but also all other pathogens (such as e.g. fungi or infection-triggering organisms, such as trypanosomes, nematodes etc.). “Antigens” of a pathogen are substances (e.g. proteins, peptides, nucleic acids or fragments thereof) of the pathogen which are capable of triggering the formation of antibodies. Antigens from a tumour are likewise encompassed by the invention. This is to be understood as meaning that the antigen is expressed in cells associated with a tumour. Antigens from tumours are, in particular, chose which are produced in the degenerated cells themselves. These are preferably antigens located on the surface of the cells. Furthermore, however, antigens from tumours are also those which are expressed in cells which are (were) not themselves (or originally not themselves) degenerated but are associated with the tumour in question. These also include e.g. antigens which are connected with tumour-supplying vessels or (re)formation thereof, in particular those antigens which are associated with neovascularization or angiogenesis, e.g. growth factors, such as VEGF, bFGF etc. Such antigens connected with a tumour furthermore include those from cells of the tissue embedding the tumour.

“Cytokine” quite generally is to be understood as meaning a protein which influences the behaviour of cells. The action of cytokines takes place via specific receptors on their target cells. Cytokines include, for example, monokines, lymphokines or also interleukins, interferons, immunoglobulins and chemokines. According to the invention, GM-CSF or G-CSF or M-CSF is particularly preferred as the cytokine.

“Administration” of the mRNA according to the invention and the cytokine or the cytokine mRNA or the adjuvo-viral mRNA or the CpG DNA or the adjuvant RNA means supplying to the organism, preferably mammal, particularly preferably human, to be treated a suitable dose of the mRNA according to the invention or of the cytokine or of the cytokine mRNA or of the adjuvo-viral mRNA or of the CpG DNA or of the adjuvant RNA. The administration can take place in any suitable manner, preferably via an injection, parenterally, e.g. intravenously, intradermally, subcutaneously, intramuscularly, intraperitoneally or intradermally. A topical or oral administration is likewise possible. The dosage of the mRNA according to the invention and of the cytokine and of the cytokine mRNA and of the adjuvo-viral mRNA and of the CpG DNA and of the adjuvant RNA has already been discussed above in more detail. Typically, the mRNA according to the invention administered or the adjuvant according to method step (b.) is in liquid form, typically in aqueous solution, which can be buffered, e.g. with phosphate buffer, HEPES, citrate, acetate etc., e.g. to a pH of between 5.0 and 5.0, in particular 6.5 and 7.5, and can contain further advantageous medicament auxiliaries and additives (e.g. human serum albumin, polysorbate 80, sugars etc.) or also salts, e.g. NaCl, KCl etc.

The present invention consequently likewise includes a method for treatment of diseases, in particular cancer or tumour diseases as well, as viral and bacterial infections, such as, for example, hepatitis B, HIV or MDR (multi-drug resistance, infections and a vaccination for prevention of the abovementioned diseases, which comprises administration of the mRNA according to the invention and at least one component of the following categories of cytokine, cytokine mRNA, adjuvo-viral mRNA, CpG DNA and/or adjuvant RNA to an organism or to a patient, in particular a human or a domestic pet. This is a combination therapy in which the mRNA according to the invention and cytokine or cytokine mRNA or adjuvo-viral mRNA or CpG DNA or adjuvant RNA are administered according to the invention together (in a mixture) separately and at the same time or separately and at staggered times.

In a preferred embodiment of the method according to the invention, the mRNA according to the invention and cytokine or cytokine mRNA or adjuvo-viral mRNA or CpG DNA or the adjuvant RNA are administered separately or at staggered times. In a particularly preferred embodiment, in the method according to the invention step b. is carried out here 1 minute to 48 hours, preferably 20 minutes to 36 hours, equally preferably 30 minutes to 24 hours, more preferably 10 hours to 30 hours, most preferably 12 hours to 23 hours, especially preferably 20 to 26 hours after step a. According to the invention, however, the cytokine or the cytokine mRNA or adjuvo-viral mRNA or the CpG DNA or the adjuvant RNA can also be administered before or simultaneously with the mRNA according to the invention.

In particular, the substances which can be employed according to method step b. can also be administered in any desired combination, i.e. according to the invention e.g. a cytokine mRNA can be administered in a mixture with an adjuvant PNA and/or a CpG DNA. If the combination of the components according to method step b. is not to take place in a mixture, the components combined with one another can also be administered separately according to method step b. It is also preferable to combine (in a mixture or separately) two or more, preferably 2-4 components of the same category, e.g. at least two different cytokines or at least two different cytokine mRNAs, with one another in method step b., optionally also, as disclosed above, with components of further categories.

In a further preferred embodiment, at least one RNase inhibitor, preferably RNasin or aurintricarboxylic acid, is additionally administered in step a, and/or b. in the method according to the invention. This serves to prevent degradation of the DNA by RNases (RNA-degrading enzymes). Such an inhibitor is typically Incorporated into the at least one composition administered according to method step (b.).

In a preferred embodiment, an immune response to an mRNA according to the invention is intensified or modulated, particularly preferably modified from a Th2 immune response into a Th1 immune response, in the method according to the invention.

In a preferred embodiment of the invention, the at least one mRNA according to the invention from step (a.) of the method according to the invention contains a region which codes for at least one antigen from a tumour chosen from the group consisting of 707-AP, AFP, ART-4, BAGE, β-catenine/m, Bcr-abl, CAMEL, CAR-1, CASF-8, CDC27/m, CDrM/m, CEA, CHV opt a, CT, Cyp-B, DAM, EGFRI, ELF2M, ETV6-AML1, G250, GAGE, GnT-V, Gp100, HAGE, HBS, HEP-2/neu, HLA-A*0201-R170I, HPV-E7, HSP70-2M, HAST-2, hTERT for hTRT), influenza matrix protein, in particular influenza A matrix M1 protein or influenza B matrix M1 protein, iCE, KIAA0205, LAGE, e.g. LAGE-1, LDLR/FUT, MAGE, e.g. MAGE-A, MAGE-B, MAGE-C, MAGE-A1, MAGE-2, MAGE-3, MAGE-6, MAGE-10, MART-1/melan-A, MC1R, myosine/m, MUC1, MUM-1, -2, -3, NA88-A, NY-ESO-1, p190 minor bcr-ab1, PmT/RARα, PRAME, proteinase 3, PSA, PSM, PTPR21, RAGE, RU1 or RU2, SAGE, SART-1 or SART-3, SEC61G, SOX9, SPC1, SSX, survivin, TEL/AML1, TERT, TNG, TPI/m, TRP-1, TRP-2, TRP-2/INT2, tyrosinase and WT1.

The at least one mRNA according to the invention particularly preferably contains a region which codes for at least one antigen from a tumour chosen from the group consisting of MAGE-A1 [accession number M77481], MAGE-A6 [accession number NM_{—}005363], melan-A [accession number NM_{—}005311], GP100 [accession number M77348], tyrosinase [accession number NM_{—}00372], survivin [accession number AF077350], CEA [accession number NM_{—}004363], Her-2/neu [accession number M11730], mucin-1 [accession number NM_{—}002456], TERT [accession number NM_{—}003219], PR3 [accession number NM_{—}002777], WT1 [accession number NM_{—}000378], PRAME [accession number NM_{—}006115], TNC (tenascin C) [accession number X78563], EGFEI (epidermal growth factor receptor 15 [accession number AG288733], SOX9 [accession number 246629], SEC61G [accession number NM_{—}014302], PTPRZ1 (protein tyrosine phosphatase, receptor type, Z-polypeptide 1) [accession number NM_{—}002851], CMV pp65 [accession number M15120], HBS antigen [accession number E00121], influents A matrix M1 protein accession number AF348197 and influenza B matrix M1 protein accession number V01099.

In the context of the present invention, the cytokine mRNA contains a section which codes for the cytokine, and the adjuvo-viral mRNA contains a section which codes for a viral protein having an adjuvant action. Nevertheless, in this case also (as also in the case of the mRNA according to the invention), the nucleotide sequence employed and called here cytokine mRNA or adjuvo-viral. mRNA can contain, in addition to the coding section, at least one further functional section, e.g. specific signal or regulation sections. These signal or regulation sections serve e.g. for better translation of the mRNA administered in the content of this invention (e.g. in a 3′ terminal untranslated region of the mRNA). Nevertheless, a signal or regulation section can also be provided in the coding region of the mRNA, e.g. 3′ or 5′ terminal region of the coding sequence, so that the signal or regulation action first occurs at the level of the expressed (fusion) protein. Thus e.g. a signal peptide sequence (e.g. a leader sequence) which—after administration, entry into the cell and expression—leads to a targeted secretion from the cell of the protein coded by the mRNA administered (mRNA according to the invention or an mRNA having an adjuvant action from method step (b.)) could be co-expressed in the coding region of the mRNA. For example, the secretion signal peptides of corresponding peptide or protein hormones (e.g. of insulin, vasopressin, glucagon etc.) or e.g. also the secretion signals of antibodies can be employed as secretion signals, in that the mRNA contains the particular nucleotide sequence thereof.

Functional fragments and/or functional variants of an mRNA according to the invention or of an antigen or of a cytokine or of a cytokine mRNA or of an adjuvo-viral mRNA or of a CpG DNA or of an adjuvant RNA of the invention are likewise encompassed according to the invention. In the context of the invention, “functional” means that the antigen or the mRNA according to the invention has immunological or immunogenic activity, in particular triggers an immune response in an organism in which it is foreign. The mRNA according to the invention is functional if it can be translated into a functional antigen (or a fragment thereof).

A “fragment” in the context of the invention is to be understood as meaning a shortened antigen or a shortened mRNA or a shortened cytokine or a shortened cytokine mRNA or an adjuvo-viral mRNA or a shortened CpG DNA or a shortened adjuvant RNA of the present invention. These can be N-terminally, C-terminally or intrasequentially shortened amino acid or nucleic acid sequences.

The preparation of fragments according to the invention is well-known in the prior art and can be carried out by a person skilled in the art using standard methods (see e.g. Maniatis et al. (2001), Molecular Cloning: Laboratory Manual, Cold Spring harbour Laboratory Press). In general, the preparation of the fragments according to the invention can be carried out by modification of the DNA sequence which codes the wild-type molecule, followed by a transformation of this DNA sequence into a suitable host and expression of this modified DNA sequence, with the proviso that the modification of the DNA dos not destroy the functional activities described. In the case of the mRNA according to the invention or a cytokine mRNA or an adjuvo-viral mRNA, the preparation of the fragment can likewise be carried out by modification of the wild-type DNA sequence, followed by an in vitro transcription and isolation of the mRNA, likewise with the proviso that the modification of the DNA does not destroy the functional activity of the particular mRNA. A fragment according to the invention can be identified, for example, via a sequencing of the fragment and a subsequent comparison of the sequence obtained with the wild-type sequence. The sequencing can be carried out with the aid of standard methods, which are numerous and well-known in the prior art.

In particular, those mRNAs according to the invention or cytokines or cytokine mRNAs or adjuvo-viral mRNAs which contain sequence differences with respect to the corresponding wild-type sequences are called “variants” in the context of the invention. These sequence deviations can be one or more insertion(s), deletion(s) and/or substitution(s) of amino acids or nucleic acids, a sequence homology of at least 60%, preferably 70%, more preferably 80%, equally more preferably 83%, even more preferably 90% and most preferably 97% existing.

In order to determine the percentage to which two nucleic acid or amino acid sequences are identical, the sequences can be aligned in order to be subsequently compared with one another. For this, e.g. gaps can be inserted into the sequence of the first amino acid or nucleic acid sequence and the amino acids or nucleic acids at the corresponding position of the second amino acid or nucleic acid sequence can be compared. If a position in the first, amino acid sequence is occupied by the same amino acid or the same nucleic acid as is the case at a position in the second sequence, the two sequences are identical at this position. The percentage to which two sequences are identical is a function of the number of identical positions divided try the total number of positions.

The percentage to which two sequences are identical can be determined with the aid of a mathematical algorithm. A preferred, but not limiting, example of a mathematical algorithm which can be used for comparison of two sequences is the algorithm of Karlin et al. (1993), PNAS USA, 90:5873-5877. Such an algorithm is integrated in the NBLAST program, with which sequences which are identical to the sequences of the present invention to a desired extent can be identified. In order to obtain a gapped alignment, as described above, the Gapped BLAST program, can be used, as is described in Altschul et al. (1997), Nucleic Acids Res, 25:3389-3402.

Functional variants in the context of the invention can preferably be mRNA molecules according to the invention or cytokine mRNA or adjuvo-viral mRNA molecules, which have an increased stability and/or translation rate compared with their wild-type molecules. There can likewise be better transport into the cell of the Ghost; organism.

Those amino acid sequences which have conservative substitution compared with the physiological sequences in particular fail under the term, variants. Those substitutions in which amino acids which originate from the same class are exchanged for one another are called conservative substitutions. In particular, there are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterised by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. A modification to a three-dimensional structure by insertion(s) or deletion(s) can easily be checked e.g. with the aid of CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Variants in which a codon usage takes place are likewise included. Each amino acid is coded by a codon which is defined by in each case three nucleotides (triplet). It is possible for a codon which codes a particular amino acid to be exchanged for another codon which codes the same amino acid. The stability of the mRNA according to the invention can be increased, for example, by choice of suitable alternative codons. This is discussed in still more detail in the following.

Suitable methods for the preparation of variants according to the invention having amino acid sequences which have substitutions compared with the wild-type sequences are disclosed e.g. in the publications U.S. Pat. No. 4,737,462, U.S. Pat. No. 4,538,585, U.S. Pat. No. 4,959,314, U.S. Pat. No. 5,116,943, U.S. Pat. No. 4,879,111 and U.S. Pat. No. 5,017,691. The preparation of variants in general is also described, in particular, by Maniatis et al, (2001), Molecular Cloning: A Laboratory Manual, Cold Spring Harbor Laboratory Press). Codons can be omitted, supplemented or exchanged here. Variants in the context of the invention can likewise be prepared by introducing into the nucleic acids which code for the variants modifications such as e.g. insertions, deletions and/or substitutions of one or more nucleotides. Numerous processes for such modifications of nucleic acid sequences are known in the prior art. One of the most used techniques is oligonucleotide-directed site-specific mutagenesis (see Comack B., Current Protocols in Molecular Biology, 8.01-8.5.9, Ausubel F. et al., ed. 1991). In this technique, an oligonucleotide is synthesized the sequence of which has a certain mutation. This oligonucleotide is then hybridised with a template which contains the wild-type nucleic acid sequence. A single-stranded template is preferably used in this technique. After annealing of the oligonucleotide and template, a DNA-dependent DNA polymerase is employed in order to synthesize the second strand of the oligonucleotide, which is complementary to the template DNA strand. As a result, a heteroduplex molecule which contains a mis-pairing formed by the abovementioned mutation in the oligonucleotide is obtained. The oligonucleotide sequence is inserted into a suitable plasmid, this is inserted into a host cell and the oligonucleotide DNA is replicated in this host cell. Nucleic acid sequences with targeted modifications (mutations) which can be used for the preparation of variants according to the invention are obtained by this technique.

In a preferred embodiment of the method according to the invention, the at least one cytokine (from the cytokine category) is chosen from the group which consists of IL-1 (α/β), IL-2, IL-3, IL-4, IL-5, IL-6, IL-7, IL-8, IL-9, IL-10, IL-12, IL-13, IL-15, IL-18, IL-21, IL-22, IL-23, IFN-α, IFN-β, IFN-γ, LT-α, MCAF, RANTES, TGFα, TGFβ1, TGFβ2, TNFα, TNFβ and particularly preferably G-CSF, M-CSF or GM-CSF, in particular (recombinant or non-recombinant) human forms of the abovementioned cytokines, as wells as variants or fragments thereof. In another preferred embodiment, cytokine mRNA which codes for one of the abovementioned cytokines or fragments or variants thereof or contains corresponding coding sections is employed in a method step b.

The mRNA from step (a.) and/or step (b.) (i.e. that, according to the invention, the cytokine or the adjuvo-viral mRNA; or the adjuvant RNA from step (b.) of the method according to the invention can be in the naked (m)RNA form or complexed with further components.

In a preferred embodiment, the mRNA from step (a.) and/or step (b,; or the adjuvant RNA from step (b.) of the method according to the invention can be in the form of modified (m)RNA, in particular stabilised (m) RNA. Modifications of the mRNA according to the Invention or of the (m)RNA from step (b.) serve here above all to increase the stability of the mRNA according to the invention or of the (m)RNA from step (b.), but also to improve the transfer of the mRNA according to the invention or of the (m)RNA from step (b.) (i.e. the cytokine mRNA, the adjuvo-viral mRNA and the adjuvant RNA) into a cell or a tissue of an organism. Preferably the mRNA according to the invention or the (m)RNA from, step (b.) of the method according to the invention has one or more modifications, in particular chemical modifications, which contribute towards increasing the half-life of the mRNA according to the invention or of the (m)RNA from step (b.) in the organism or improving the transfer of the mRNA according to the invention or of the (sp RNA from step (b.) into the cell or a tissue.

In a particularly preferred embodiment of the present invention, the G/C content of the coding region of the modified mRNA according to the invention from step (a.) and/or of the cytokine mRNA and/or of the adjuvo-viral mRNA from step (b.) of the method according to the invention is increased compared with the G/C content of the coding region of the particular wild-type RNA, the coded amino acid sequence of the modified mRNA according to the invention or of the mRNA from step (b.) preferably not being modified compared with the coded amino acid sequence of the particular wild-type mRNA.

This modification is based on the fact that the sequence of the mRNA region to be translated is important rot efficient translation of an mRNA. The composition and the sequence of the various nucleotides is of significance here. In particular, sequences having an increased G (guanosine)/C (cytosine) content are more stable than sequences having an increased A (adenosine)/U (uracil) content. According to the invention, the codons are therefore varied compared with the wild-type mRNA, while retaining the translated amino acid sequence, such that they include an increased amount of G/C nucleotides. On the basis of the fact that several codons code for one and the same amino acid (so-called degeneration of the genetic code), the most favourable codons for the stability can be determined (so-called alternative codon usage).

Depending on the amino acid to be coded by the modified mRNA (from step (a.) or (b.)), there are various possibilities for modification of the mRNA sequence according to the invention or the cytokine mRNA sequence or the adjuvo-viral mRNA sequence compared with the wild-type sequence. In the case of amino acids which are coded by codons which contain exclusively G or C nucleotides, no modification of the codon is necessary. Thus, the codons for Pro (CCC or CCG), Arg (CGC or CGG), Ala (GGG or GCG) and Gly (GGC or GGG) require no modification, since no A or U is present.

In contrast, codons which contain A and/or U nucleotides can be modified by substitution of other codons which code the same amino acids but contain no A and/or U. Examples of these are:
the codons for Pro can be modified frees CCG or GGG to CGC or GCG;
the codons for Arg can be modified from GGG or CGA or GGG or GGG to CGC or GGG;
the codons for Ala can be modified from GCD or GCG to GCG or GCG;
the codons for Gly can be modified from GGG or GGA to GGC or GGG.

In other cases, although A or U nucleotides cannot be eliminated from the codons, it is however possible to decrease the A and U content by using codons which contain a lower content of A and/or U nucleotides. Examples of these are:
the codons for Phe can be modified from UUU to GGG;
the codons for Leu can be modified from UUA, GOG, CGG or CUA to CUC or CUG;
the codons for Ser can be modified from GCU or UCA or AGU to UCC, OCG or AGO;
the codon for Tyr can be modified from GAG to GAG;
the codon for Cys can be modified from UGU to GGC; the codon for His can be modified from CAU to CAC;
the codon for Gln can be modified from CAA to GAG;
the codons for Ile can be modified from Add or AUA to AUC;
the codons for Thr can be modified from ACU or ACA to ACC or ACG;
the codon for Asn can be specified from AAU to AAC;
the codon for Lys can be modified from AAA to AAG;
the codons for Val can be modified from GUU or GUA to GUC or GUG;
the coders for Asp can be modified from GAU to GAG;
the codon for Glu can be modified from GAA to GAG;
the stop codon UAA can be modified to DAG or UGA.

In the case of the codons for Met (AUG) and Trp (uGG), on the other hand, there is no possibility of sequence modification.

The substitutions listed above can be used either individually or in all possible combinations to increase the G/C content of the modified mRNA according to the invention or of the cytokine mRNA or of the adjuvo-viral mRNA compared with the particular wild-type mRNA (of the original sequence). Thus, for example, ail codons for Thr occurring in the wild-type sequence can be modified to ACC (or ACG). Preferably, however, for example, combinations of the above substitution possibilities are used:
substitution of all codons coding for Thr in the original sequence (wild-type mRNA) to ACC (or ACG) and substitution of all codons originally coding for Ser to UCC (or UCG or AGC);
substitution of: all codons coding for Ile in toe original sequence to AUG and substitution of all codons originally coding for Lys to AAG and substitution of all codons originally coding for Tyr to UAC;
substitution of all codons coding for Val in the original sequence to GUC (or GUG) and substitution of all codons originally coding for Glu to GAG and substitution of all codons originally coding for Ala to GGC (or GCG) and substitution of all codons originally coding for Arg to CGC (or CGG);
substitution of all codons coding for Val in the original sequence to GUC (or GUG) and substitution of all codons originally coding for Glu to GAG and substitution of all codons originally coding for Ala to GCG (or GCG) and substitution of all codons originally ceding for Gly to GGC (or GGG) and substitution of all codons originally coding for Asn to AAC;
substitution of all codons coding for Val in the original sequence to GUC (or GUG) and substitution of all codons originally coding for Phe to DUG and substitution of all codons originally coding for Cys to UGC and substitution of all codons originally coding for Leu to CUG (or CUC) and substitution of all codons originally coding for Gln to CAG and substitution of all codons originally coding for Pro to CCC (or CCG); etc.

Preferably, the G/C content of the antigen-coding region of the modified mRNA according to the invention or of the cytokine mRNA or of the adjuvo-viral mRNA is increased by at least 7% points, more preferably by at least 15% points, particularly preferably by at least 20% points, compared with the G/C content of the coded region of the wild-type mRNA which codes for the antigen.

In this connection, it is particularly preferable to increase to the maximum the G/C content of the modified mRNA according to the invention or of the cytokine mRNA or of the adjuvo-viral mRNA, in particular in the region coding for the antigen, compared with the wild-type sequence.

A further preferred modification of the mRNA from step (a.) and/or step (b.) of the method according to the invention is based on the finding that the translation efficiency is likewise determined by a different frequency in the occurrence of tRNAs in cells. Thus, if so-called “rare” codons are present in an RNA sequence to an increased extent, the corresponding mRNA is translated to a significantly poorer degree than in the case where codons which code for relatively “frequent” tRNAs are present.

In the modified mRNA according to the invention or the cytokine mRNA or the cytokine mRNA or the adjuvo-viral mRNA of the method according to the invention, the region which codes for the antigen is thus modified compared with the corresponding region of the wild-type mRNA such that at least one codon of the wild-type sequence which codes for a tRNA which is relatively rare in the cell is exchanged for a codon which codes for a tRNA which is relatively frequent in the cell and carries the same amino acid as the relatively rare tRNA. By this modification, the RNA sequences are modified such that codons for which frequently occurring tRNAs are available are inserted. In other words, according to the invention, by this modification all codons of the wild-type sequence which code for a tRNA which is relatively rare in the cell can in each case be exchanged for a codon which codes for a tRNA which is relatively frequent in the cell and which in each case carries the same amino acid as the relatively rare tRNA.

Which tRNAs occur relatively frequently in the cell and which, in contrast, occur relatively rarely is known to a person skilled in the art; cf. e.g. Akashi, Curr. Opin. Genet. Dev. 2001, 11(6): 650-666. The codons which use for the particular amino acid the tRNA which occurs the most frequently, that is to say e.g. the Gly codon, which uses the tRNA which occurs the most frequently in the (human; cell, are particularly preferred.

It is particularly preferable according to the invention to link the sequential G/C content which is increased, in particular the maximum such content, in the modified mRNA according to the invention or the cytokine mRNA or the adjuvo-viral mRNA with the “frequent” codons without modifying the amino acid sequence of the antigen coded by the coding region of the mRNA. This preferred embodiment provides a particularly efficiently translated and stabilised mRNA according to the invention, e.g. for the method according to the invention.

The determination of an mRNA according to the invention modified as described above (increase in the G/C content; exchange of tRNAs) can be carried out with the aid of the computer program explained in WO 02/098443—the disclosure content of which is included in its full scope in the present invention. Kith this computer program, the nucleotide sequence of any desired mRNA can be modified with the aid of the genetic code or the degenerative nature thereof such that a maximum G/C content results, in combination with the use of codons which code for tRNAs occurring as frequently as possible in the cell, the amino acid sequence coded by the modified mRNA preferably not being modified compared with the non-modified sequence. Alternatively, it is also possible to modify only the G/C content or only the codon usage compared with the original sequence. The source code in Visual. Basic 6.0 (development environment used; Microsoft Visual Studio Enterprise 6.0 with Servicepack 3) is likewise described in WO 02/098443.

In a further preferred embodiment of the present invention, the A/U content in the environment of the ribosome binding site of the modified mRNA from step (a.) and/or step (b.) of the method according to the invention is increased compared with the A/U content in the environment of the ribosome binding site of the particular wild-type mRNA. This modification (an increased A/U content around the ribosome binding site) increases the efficiency of ribosome binding to the mRNA according to the invention. An effective binding of the ribosomes to the ribosome binding site (Kozak sequence: GCCGCCACCAUGG, the ANTS forms the start codon) in turn has the effect of an efficient translation of the mRNA according to the invention or of the other above-mentioned mRNAs having adjuvant properties.

An embodiment of the present invention which is likewise preferred relates to a method according to the invention, wherein she coding region and/or the 5′ and/or 3′ untranslated region of the mRNA from step (a.) and/or step (b.) (i.e. cytokine mRNA or adjuvo-viral mRNA) is modified compared with the particular wild-type mRNA such that is contains no destabilizing sequence elements, the coded amino acid sequence of the modified mRNA preferably not being modified compared with the particular wild-type mRNA. It is known that, for example, in the sequences of eukaryotic mRNAs destabilizing sequence elements (DSE) occur, to which signal proteins bind and regulate the enzymatic degradation of the mRNA in vivo. For further stabilisation of the modified mRNA optionally in the region which codes for the antigen, one or more such modifications compared with the corresponding region of the wild-type mRNA can therefore be carried out, so that no or substantially no destabilizing sequence elements are contained there.

According to the invention, DSE present, in the untranslated regions (3′-end/or 5′-UTR) can likewise be eliminated from the mRNA according to the invention by such modifications.

Such destabilizing sequences are e.g. All-rich sequences (AURES), which occur in 3′-UTR sections of numerous unstable mRNAs (Caput et al., Proc. Natl. Acad. Sci. USA 1986, 83: 1670 to 1674). The mRNA molecules according to the invention or adjuvant mRNA molecules contained in the method according to the invention are therefore preferably modified compared with the wild-type mRNA such that they contain no such destabilizing sequences. This also applies to those sequence motifs which are recognized by possible endonucleases, e.g. the sequence GAACAAG, which is contained in the 3′-UTR segment of the gene which codes for the transferrin receptor (Binder et al., EMBO J. 1994, 13: 1969 to 1980). These sequence motifs are also preferably removed in the modified mRNA according to the invention or the adjuvant. mRNA (cytokine mRNA or adjuvo-viral mRNA) of the method according to the invention.

In a further preferred embodiment of the present invention, the mRNA from step (a.) and/or step (b.) (e.g. the cytokine mRNA) of the method according to the invention has a 5′ cap structure. Examples of cap structures which can be used according to the invention are m7G(5′)ppp (5′(A,G(5′)ppp(5′)A and G(5′)ppp(5′)G. Such modifications can also occur in the adjuvant RNA from step (b.).

It is furthermore preferable for the mRNA from step (a.) and/or step (b.) of the method according to the invention to have, in a modified form, a poly(A) tail, preferably of at least 25 nucleotides, more preferably of at least 50 nucleotides, even more preferably of at Least 70 nucleotides, equally more preferably of at least 100 nucleotides, most preferably of at least 200 nucleotides.

Likewise preferably, the mRNA from step (a.) and/or step (b.) of the method according to the invention has, in a modified form, at least one IRES and/or at least one 5′ and/or 3′ stabilising sequence. According to the invention, one or more so-called IRES (internal ribosomal entry site) can accordingly be inserted into the mRNA from step (a.) and/or step (b.). An IRES can thus function as the sole ribosome binding site, but it can also serve to provide an mRNA from step (a.) and/or step (b.) which codes several antigens which are to be translated by the ribosomes independently of one another (multicistronic mRNA). Examples of IRES sequences which can be used, according to the invention are those from picornaviruses (e.g. FMDV), pestiviruses (CFFV), polioviruses (PV), encephalomyocarditis viruses (ECMV), foot and mouth disease viruses (FHDV), hepatitis C viruses (HCV), classical swine fever viruses (CSFV), mouse leukoma virus (MLV), simian immunodeficiency viruses (SIV) or cricket paralysis viruses (CrPv).

The mRNA from step (a.) and/or step (b.) of the method according to the invention furthermore preferably has at least one 5′ and/or 3′ stabilising sequence. These stabilizing sequences in the 5′ and/or 3′ untranslated regions have the effect, of increasing the half-life of the mRNA according to the invention in the cytosol. These stabilising sequences can have a 100% sequence homology to naturally occurring sequences which occur in viruses, bacteria and eukaryotes, but can also be partly or completely synthetic in nature. The untranslated sequences (UTR) of the β-globin gene, e.g. from Heme sapiens or Xenopus laevis may be mentioned as an example of stabilizing sequence which can be used in the present invention. Another example of a stabilising sequence has the general formula (C/U)CCANₓCCC(U/A)PyₓUC(C/U)CC, which is contained in the 3′UTR of the very stable mRNA which codes for α-globin, α-(I)-collagen, 15-lipoxygenase or for tyrosine hydroxylase (cf. Holcik et al., Proc. Natl. Acad. Sci. USA 1.397, 34: 2410 to 2414). Such stabilising sequences can of course be used individually or in combination with one another and also in combination with other stabilising sequences known to a person skilled in the art. The mRNA from step (a.) and/or step (b.) of the method according to the invention is therefore preferably present as globin UTR (untranslated regions)-stabilised mRNA, in particular as β-globin UTR-stabilized mRNA. It has been found, according to the invention, that injection of naked β-globin UTR (untranslated regions)-stabilised mRNA according to the invention, optionally in combination with adjuvant mRNA likewise modified in such a manner or otherwise, into the ear pinna of a mammal (e.g. of mice) induces a specific immune response to the antigen which is coded by the mRNA according to the invention (17). In other words, the inventors have monitored and investigated the course of the injected β-globin UTR-stabilized mRNA and the type of immune response which it triggers and have thus detected a translation in vivo (see FIG. 1). This vaccination strategy has been investigated further, and a pharmaceutical mRNA which can be used in human clinical trials has been developed.

In a preferred embodiment of the present invention, the modified mRNA from step (a.) and/or step (b.) or the adjuvant RNA from step (b.) of the method according to the invention contains at least one analogue of naturally occurring nucleotides. This/these analogue/analogues serves/serve for further stabilising of the modified mRNA according to the invention, this being based on the fact that the RNA-degrading enzymes occurring in the cells preferentially recognize naturally occurring nucleotides as a substrate. The degradation of RNA can therefore be made difficult by insertion of nucleotide analogues into the RNA, whereby the effect on the translation efficiency on insertion of these analogues, in particular in the coding region of the mRNA, can have a positive or negative effect on the translation efficiency. In a list which is in no way conclusive, examples which may be mentioned of nucleotide analogues which can be used according to the invention are phosphoroamidates, phosohorothioates, peptide nucleotides, methylphosphoxiates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642. According to the invention, such analogues can occur in untranslated and translated regions of the modified mRNA.

Various methods for carrying out the modifications described, are familiar to a person skilled in the art. Some of these methods have already been described in the above section on the variants of the invention. For example, for substitution of codons in the modified mRNA according to the invention or an mRNA (cytokine mRNA or adjuvo-viral mRNA; or adjuvant RNA from seep (b.) or in the case of shorter coding regions, the entire mRNA according to the invention can be synthesized chemically using standard techniques.

Nevertheless substitutions, additions or eliminations of bases are preferably inserted, using a DNA matrix for the preparation of the modified mRNA according to the invention or an mRNA from step (b,) with the aid of techniques of the usual targeted mutagenesis (see e.g. Maniatis et al., Molecular Cloning: A Laboratory Manual, Cold Spring Harbor Laboratory Press, 3rd ed., Cold Spring Harbor, N.Y., 2001), In such a process, for the preparation of the mRNA according to the invention or an mRNA from step (b.), a corresponding DNA molecule is transcribed in vitro. This DNA matrix has a suitable promoter, e.g. a T7 or SP6 promoter, for the in vitro transcription, which is followed by the desired nucleotide sequence for the mRNA (according to the invention) to be prepared and a termination signal for the in vitro transcription. According to the invention, the DNA molecule which forms the matrix of the RNA construct to be prepared is prepared by fermentative proliferation and subsequent isolation as part of a plasmid which can be replicated in bacteria. Plasmids which may be mentioned as suitable for the present invention are e.g. the plasmids pT7Ts (GenBank accession number U26404; Lai et al., Development 1995, 121: 23-19 to 2360), pGEM® series, e.g. pGEM®-1 (GenBank accession number X65300; from Promega) and pSPS4 (GenBank accession number X65327); cf. also Mezei and Storts, Purification of PCR Products, in: Griffin and Griffin (ed.), PCR Technology: Current Innovation, CRC Press, Boca Raton, Fla., 2001.

Using short synthetic DNA oligonucleotides which contain short single-stranded extensions at the cleavage sites formed, or genes prepared by chemical synthesis, the desired nucleotide sequence can thus be cloned info a suitable plasmid by molecular biology methods with which a person skilled in the art is familiar (cf. Maniatis et ai., supra). The DNA molecule is then excised out of the plasmid, in which it can be present in one or several copies, by digestion with restriction endonucleases.

In addition to the abovementioned modifications at the level of the nucleotide sequence, further modifications can be inserted into the mRNA from step a. and/or b.

In a further embodiment of the present invention, the mRNA from step (a.) and/or step (b.) or the adjuvant RNA from step (b.) of the method according to the invention is complexed or condensed and inasmuch modified with at least one cationic or polycationic agent. Such a cationic or polycationic agent is preferably an agent which is chosen from the group consisting of protamine, poly-L-lysine, poly-L-arginine and histones.

By this modification on the basis of complexing of the mRNA from step (a.) (mRNA according to the invention) and/or step (b.) or the adjuvant RNA from step (b.), the effective transfer of the modified (m)RNA into the cells to be treated or the tissue to be treated or the organism to be treated can be improved in that the abovementioned (m)RNA is associated with a cationic peptide or protein or bound thereto. In particular, the use of protamine as a polycationic, nucleic acid-binding protein is particularly effective in this context. The use of other cationic peptides or proteins, such as poly-L-lysine or histories, is of course likewise possible. This procedure for stabilising the abovementioned (m) RNA molecules in a method according to the invention is described, for example, in EE-A-1083232, the disclosure content of which in this respect is included in its full scope in the present invention.

In a further embodiment of the present invention, the modified mRNA according to the invention or the adjuvant mRNA or adjuvant RNA from step (b.) of the method according to the invention is stabilised and inasmuch modified with polyethyleneimine (PEI).

The mRNA according to the invention, the cytokine mRNA, the adjuvo-viral mRNA and/or the adjuvant RNA (in each case modified or non-modified) can be in single- or double-stranded form and can be employed as such or in a mixture in a method according to the invention. In the case of a double-stranded nature, at least one conventionally open terminus of the double strand, preferably both, can also be bonded covalently to one another, e.g. via a hairpin structure.

All the modifications described above with reference to the mRNA according to the invention from step (a.) (e.g. insertion of nucleotide analogues, 5′ cap structure etc.) are likewise used in the context of the invention on the adjuvant RNA or on the cytokine mRNA or adjuvo-viral mRNA from step (b.) of the method according to the invention.

All the modifications described above to the mRNA according to the invention or the cytokine mRNA, the adjuvo-viral mRNA or the adjuvant RNA of the method according to the invention can occur individually or in combinations with one another in the context of the invention.

The invention also provides a product comprising at least one mRNA according to the invention containing a region which codes for at least one antigen of a pathogen or at least one tumour antigen, and at least one component of at least one of the following categories chosen from the group consisting of a cytokine, a cytokine mRNA, an adjuvo-viral mRNA, a CpG DNA and an adjuvant RNA, as a combination preparation for simultaneous, separate or time-staggered use in the treatment and/or prophylaxis of tumour diseases (e.g. lymphomas, pancreas tumour, melanomas and other types of skin cancer, solid tumours of the liver, the lung, the head, the intestine, the stomach, sarcomas), allergies, autoimmune diseases, such as multiple sclerosis, viral and/or bacterial infections, in particular HIV, influenza, rubella, measles, rabies, herpes, dengue fever, yellow fever, hepatitis, pneumonias, Legionnaires' disease. Streptococci, Enterococci or Staphylococci infections or infections with protozoological pathogens, e.g. trypanosomes.

Patients having the abovementioned indications can also be treated by a method according to the invention.

The constituents of the product according to the invention; at least one mRNA according to the invention containing a region which codes for at least one antigen of a pathogen or at least one tumour antigen (1st constituent; and at least one cytokine and/or at least one cytokine mRNA and/or at least one adjuvo-viral mRNA and/or at least one CpG DNA and/or at least one adjuvant RNA (2nd constituent) are in a functional unit due to their targeted use. The constituents of the product cannot display the advantageous action according to the invention described above independently of one another, so that in spite of the spatial/physical separation of constituents 1 and 2 (for simultaneous, separate or time-staggered administration), they are used as a novel combination product which is not described in the prior art. Since constituent 2 can comprise several components, e.g. cytokine mRNA and CpG DNA or a cytokine and CpG DNA or also 2 different cytokine mRNAs, constituent 2 can be in the form of a mixture of (optionally various) components optionally of various of the abovementioned categories or the (optionally various) components optionally of various of the abovementioned categories of constituent 2 can also be present separately from one another.

A product according to the invention can comprise all the constituents, substances and embodiments such as are employed in a method or therapy method or method for treatment and/or prophylaxis of diseases or combination therapy method according to the present, invention.

The invention also provides a kit which comprises at least one mRNA according to the invention containing a region which codes for at least one antigen of a pathogen or at least one tumour antigen, and at least one component of at least one of the following categories chosen from the group consisting of a cytokine, a cytokine mRNA, an adjuvo-viral mRNA, a CpG DNA and an adjuvant RNA, the at least one mRNA according to the invention containing a region which codes for at least one antigen of a pathogen or at least one tumour antigen, and the at least one cytokine or at least one cytokine mRNA or at least one adjuvo-viral mRNA or at least, one CpG DNA or at least one adjuvant RNA being separate from one another, that is to say the kit comprises at least two parts. The kit will comprise more than two parts if, in the context of this invention, two or more adjuvant components such as can be administered e.g. in method step (b.) are contained in the kit separately from one another.

A preferred embodiment of the invention relates to the use of the kit for treatment and/or prophylaxis of cancer diseases, tumour diseases, in particular of the abovementioned specific tumour species, allergies, autoimmune diseases, such as multiple sclerosis, and/or viral and/or bacterial infections, such as, for example, hepatitis B, HIV or MDR (multi-drug resistance) infections, influenza, herpes, rubella, measles, rabies, Streptococci, Pneumococcal, Enterococci, Staphylococci or Escherichia infections or further infectious diseases mentioned in this Application.

The mRNA mentioned in the following description of the figures and in the following examples relates to the mRNA according to the invention.

### FIGURES

FIG. 1 shows the in vivo translation of injected mRNA according to the invention. Injection buffer (150 mid NaCl, 10 mM HEPES (buffer), β-galactosidase-coding β-globin UTR-stabilised mRNA, diluted in injection buffer (lac Z mRNA) or β-galactosidase-coding DNA in PBS (lac Z DNA) were injected into the ear pinna of mice. 16 hours after the injection, the mice were sacrificed and the ears were shaved, removed and frozen in embedding medium. Frozen sections were then prepared, fixed and stained overnight with solution containing X-Gal. Cells which expressed β-galactosidase appeared blue. The number of blue cells detected in each section is shown in the graphs (left half of FIG. 1). The length of the ear section analysed is plotted on the x-axis (0 is arbitrarily assigned to the first section which shows blue cells; in the mice injected with buffer, the region lying 2 mm around the injection site was analysed and the 0 determined arbitrarily): Each section is 50 μm and a few successive sections thus cover a total distance of a few millimetres. In each of the graphs (buffer-injected mice, mRNA-injected mice, DNA-injected mice), the two sections which are identified by an asterisk and a grey column are the sections which are shown in the accompanying microscope images (right half of FIG. 1). Open arrows here indicate an endogenous expression of S-galactosidase activity chiefly in the ear follicles. This endogenous activity is detectable by a very weak and diffuse blue coloration. Arrows filled in black indicate blue cells which result from uptake and translation of an exogenous nucleic acid which codes β-galactosidase. Such cells are located in the dermis at the injection site and show an intense blue coloration. Individual sections were photographed. The sections having the most blue cells are shown (they correspond to the sections marked with an asterisk in the graphs). The number; of blue cells in each of the successive sections is shown on she γ-axes in the graphs (left half of FIG. 1).

FIG. 2 shows the triggering of an antigen-specific immune response of type Th2 by the injection of mRNA. Mice were vaccinated and boosted with mRNA or DNA which codes for β-galactosidase, or they were injected with injection buffer. Two weeks later, the mice received a boost injection. Two weeks later again, the amount of β-galactosidase-specific antibodies present in the serum was determined by ELISA using isotype-specific reagents. The left half of FIG. 2 shows the IgG1 production, the right half of FIG. 2 shows the IgG2a production. ( [symbol] ) shows the curve for DNA-injected mice, ( [symbol] ) shows the curve for RNA-injected mice and ( [symbol] ) shows the curve for mice which were injected with injection buffer.

FIG. 3 shows the polarisation of a Th2 immune response into a Th1 immune response caused by the injection of GM-CSF. All the results shown relate to mice of the same group in one experiment. The total number of mice which showed an immune response in four independent experiments is shown in Table 1 (FIG. 4).

FIG. 3a: Mice were injected either with βgalactosidase, emulsified in Freund's adjuvant, or mRNA which codas for β-galactosidase, or Injection buffer has a negative control). GM-CSF (total amount of 2 μg of recombinant protein: approx, 10⁴ U (units)) were injected once, either 24 hours or 2 hours before injection of the mRNA or 24 hours after injection of the mRNA (corresponds to groups GM-CSF T-1, GM-CSG T-0 and GM-CSF T+1). The amount of βgalactosidase-specific IgG1 or IgG2a antibodies contained in the blood of the injected mice was determined by ELISA (1:10 serum dilution). The background which was chiefly obtained by the serum of buffer-injected mice at the same dilution was subtracted. The left half of FIG. 3a shows β-gal-specific IgG1 antibodies ( [symbol] ), the right half of FIG. 3a shows β-gal-specific IgG2a antibodies ( [symbol] , grey).

FIG. 3b: The in vitro reactivation of T cells by β-galactosidase was checked with the aid of a cytokine detection on day 4 of the culture. The content of IFNγ ( [symbol] ) and IL-4 ( [symbol] grey) in the supernatant of the splenocyte culture used was measured by means of ELISA.

FIG. 3c: The cytotoxic activity of splenocytes which were cultured in the presence of purified β-galactosidase for six days was checked in a chromium release assay. The target cells were P815 (H2^{d}) cells, which were either charged ( [symbol] ) with the synthetic peptide TRREARIGL, which corresponds to the dominant H2-L^{d} epitope of β-galactosidase, or were not charged (□).

FIG. 4 shows Table 1, in which the total number of mice injected is shown. The total number of s-ice whose splenocytes snowed a detectable cytokine release or a β-galactosidase-specific cytotoxic activity in vitro in independent experiments is shown. Mice in which at least 10% more TPHPARIGL-charged cells were killed; compared with the average of the cells killed in the negative control group (buffer-injected mice), were classified as mice with an immune response (responding). Splenocyte cultures which contained at least 100 pg/ml of cytokine more than the total content of cytokine in the splenocyte cultures of the negative control mice (buffer-injected mice) were classified as responding cultures (responding mice). The figures in bold indicate groups in which more than half of the mice showed an immune response to the vaccine according to the parameters investigated (cytokine or cytotoxic activity).

FIG. 5: shows the polarisation of a Th2 immune response into a Th1 immune response caused by the injection of GM-CSF RNA in addition to the mRNA according to the invention. All the results shown relate to mice of the same group in one experiment. For this, mice were injected with mRNA which codes for β-galactosidase, GM-CSF RNA or injection buffer. GM-CSF RNA (total amount 50 μg) was injected once, either 24 hours or 2 hours before injection of the mRNA or 24 hours after injection of the mRNA (corresponds to groups GM-CSF RNA T-1, GM-GSF RNA T-0 and GM-CSF RNA T+1). The amount of IFN-γ secreted which was contained in the blood of the injected mice was determined by ELISA.

FIG. 6; Graphic of the plasmid vector pT7TS.

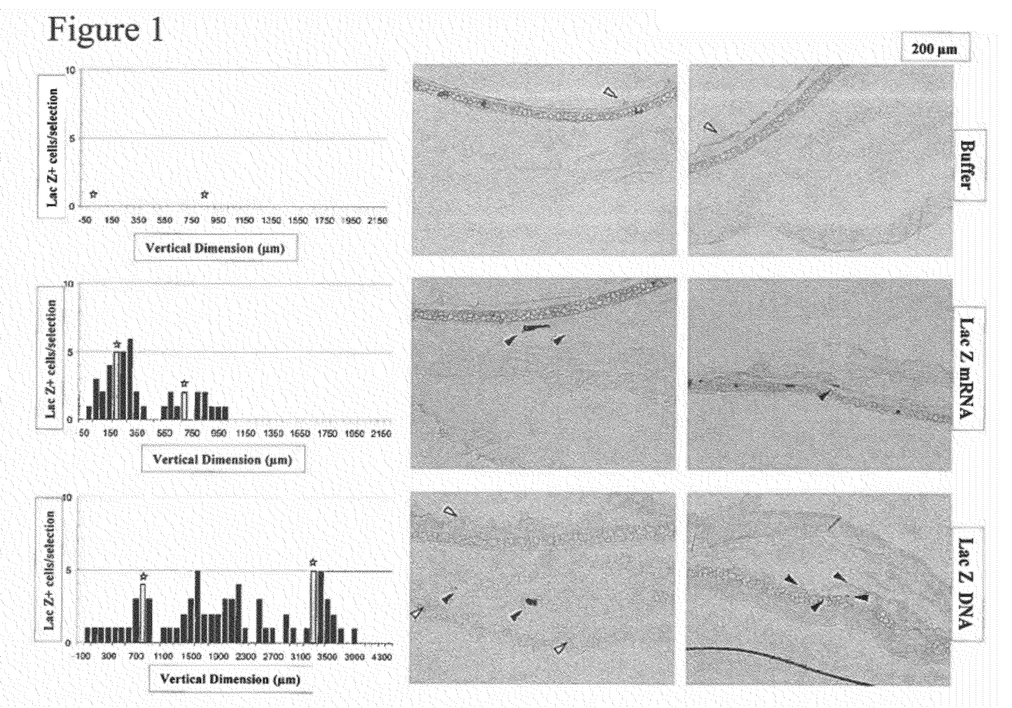

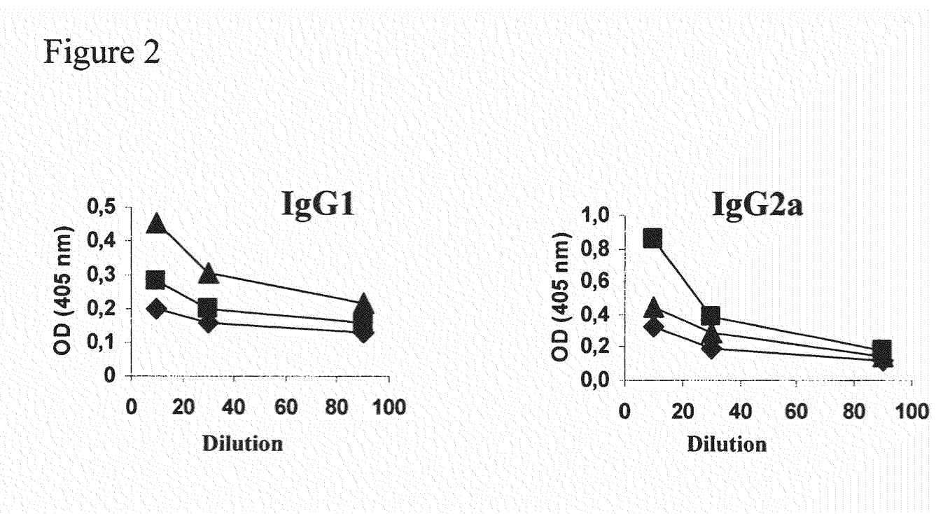

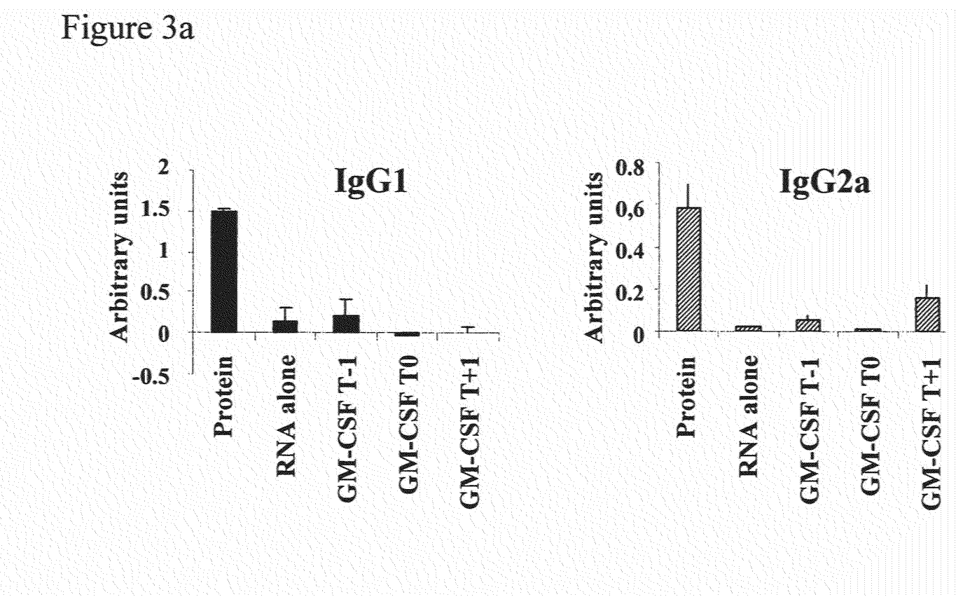

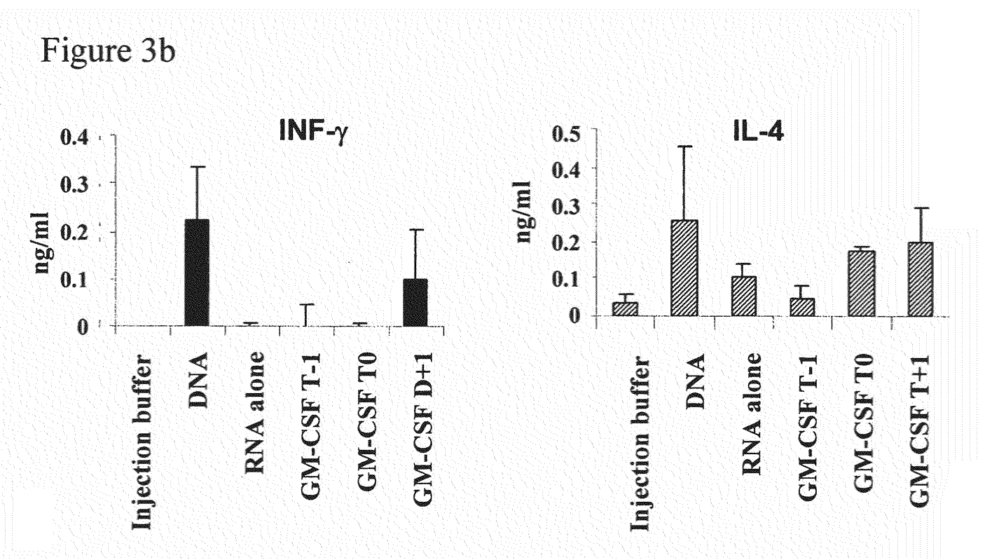

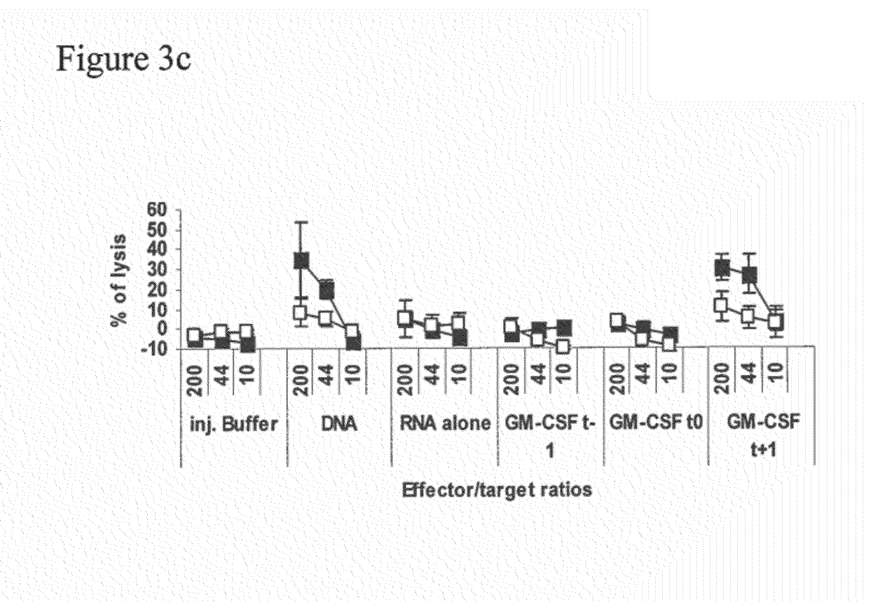

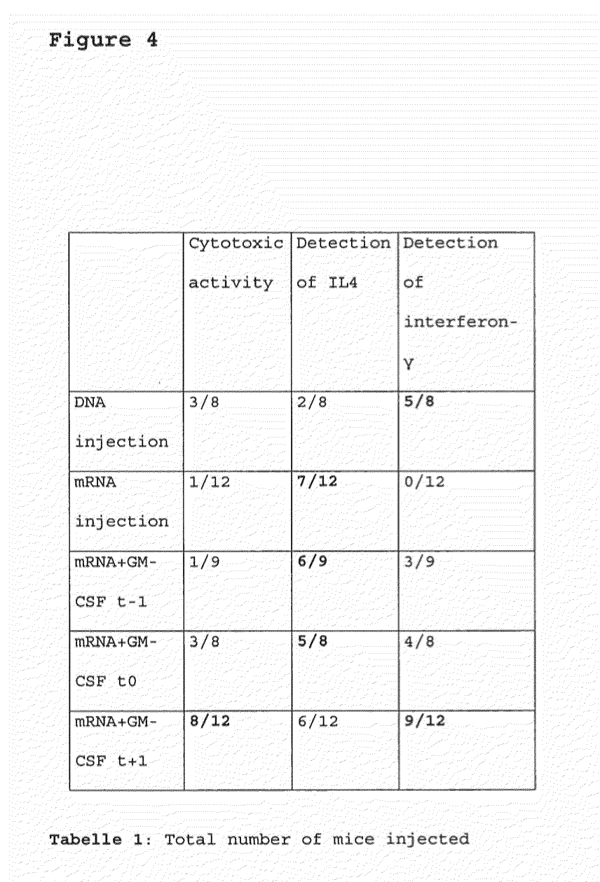

The following examples are intended to illustrate the invention further. They are not intended to limit the subject matter of the inventions thereto.

### EXAMPLES

### Example 1

### Preparation of the mRNA

The mRNA was obtained by in vitro transcription of suitable template DNA and subsequent extraction and purification of the mRNA. Standard methods which are described in numerous instances in the prior art and with which the person skilled in the art is familiar can be used for this. For example, Maniatis et al. (20015, Molecular Cloning: Laboratory Manual, Cold Spring Harbour Laboratory Dress. The same also applies to the sequencing of the mRNA, which followed the purification (described below) of the mRNA. The NBLAST program in particular was used here.

The mRNA according to the invention was generally prepared in accordance with the following procedure:

### 1. Vector

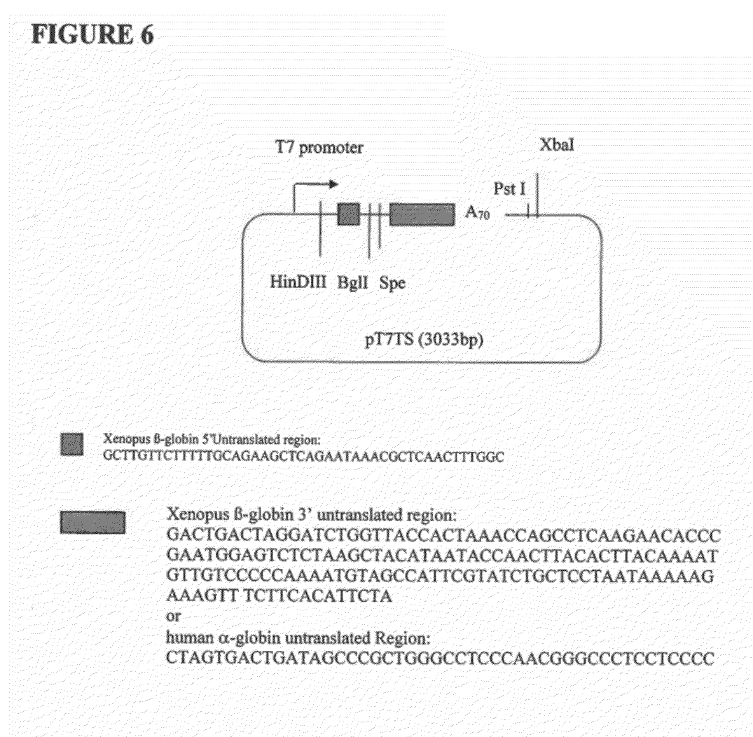

The genes for which the particular mRNA codes were inserted into the plasmid vector pT7TS. pT7TS contains untranslated regions of the alpha- or beta-globin gene and a polyA tail of 70 nucleotides (see FIG. 6):

Plasmids of high purity were obtained with the Qiagen Endo-free Maxipreparation Kit or with the Machery-Nagel GigaPrep Kit. The sequence of the vector was checked via a double-strand sequencing from the T7 promoter up to the PstI or XbaI site and documented. Plasmids in which the gene sequence cloned in was correct and without mutations were used for the in vitro transcription.

### 2. Genes

The genes for which the mRNA according to the invention codes were amplified by means of PCR or extracted from the plasmids (described above). Examples of gene constructs which were employed are

GP100 (accession number M77348):

PGR fragment SpeI in T7TS HinDIII blunt/SpeI

MAGE-A1 (accession number M77481);

plasmid fragment HinDIII/SpeI in T7TS HinDIII/SpeI

MAGE-A6 (accession number; NM_{—}005363):

PGR fragment SpeI in T7TS HinDIIIblunt/SpeI

Her2/neu (accession number: M11730):

PGR fragment HinDIII/SpeI in T7TS HinDIII/SpeI

Tyrosinase (accession number; NM_{—}000372):

plasmid fragment EcoRI blunt in T7TS HinDIII blunt/SpeI blunt

Melan-A (accession number; NM_{—}005511):

plasmid fragment NotI blunt in T7TS HindIII blunt/SpeI blunt

CEA (accession number; NM_{—}004363):

PGR fragment HinDIII/SpeI in T7TS HinDIII/SpeI

Tert (accession number; NM_{—}003219):

PGR fragment HindIII/SpeI in T7TS HinDIII/SpeI

WT1 (accession number; NM_{—}000378):

plasmid fragment EcoRV/KpnI blunt in T7TS HinDIII blunt/SpeI blunt

PR3 (accession number: NM_{—}002777):

plasmid fragment EcoRI blunt/XbaI in T7TS HinDIII blunt/SpeI

FRAME (accession number: NM_{—}006115):

plasmid fragment BamHI blunt/XbaI in T7TS HinDIII blunt/SpeI

Survivin (accession number AF077350):

PCR fragment HinDIII/SpeI in T7TS HinDIII/SpeI

Mucin1 (accession number NM_{—}002456);

plasmid fragment: SacI blunt/BamHI in T7TS HinDIII blunt/BglII

Tenascin (accession number X78565):

PGR fragment BglII blunt/SpeI in T7TS HinDIII blunt/SpeI

EGFR1 (accession number AF2887388):

PGR fragment HinDIII/SpeI in T7TS HinDIII/SpeI

Sox9 (accession number Z46629):

PGR fragment HinDIII/SpeI in T7TS HinDIII/SpeI

Sec61G (accession number NM_{—}014302):

PCR fragment HinDIII/SpeI in T7TS HinDIII/SpeI

PTRS1 (accession number NM_{—}002351):

PGR fragment EcoRV/SpeI in T7TS HinDIII blunt/SpeI

### 3. In Vitro Transcription

### 3.1. Preparation of Protein-Free DNA

500 μg of each of the plasmids described above were linearised in a volume of 2.5 ml by digestion with the restriction enzyme PstI or XbaI in a 15 ml saloon tube. This cleaved DMA construct was transferred into the RNA production unit. 2.5 ml of a mixture of phenol/chloroform/isoamyl alcohol were added to the linearised DNA. The reaction vessel was vortexed for 2 minutes and centrifuged at 4,000 rgm for 5 minutes. The aqueous phase was removed and mixed with 1.75 ml 2-propanol in a 15 ml Falcon tube. This vessel was centrifuged at 4,000 rpm for 30 minutes, the supernatant was discarded and 5 ml 75% ethane-1 were added. The reaction vessel was centrifuged at 4,000 rpm for 10 minuses and the ethanol was removed. The vessel was centrifuged for a further 2 minutes and the residues of the ethanol were removed with a microliter pipette tip. The DNA pellet was then dissolved in 500 μl RNase-free water (1 μg/μl).

3.2. Enzymatic mRNA Synthesis

### Materials:

T7 polymerase; purified from an E. coli strain which contains a plasmid with the gene for the polymerase. This RNA polymerase uses as the substrate only T7 phage promoter sequences (Fermentas),
NTPs; synthesized chemically and purified via HPLC. Purity more than 96% (Fermentas),
CAP analogue: synthesized chemically and purified via HPLC. Purity more than 90% (Trilink),
RNsse inhibitor: RNasin, injectable grade, prepared by a recombinant method (E. coli) (Fermentas),
DNaae: distributed as a medicament via pharmacies as Pulmozym® (dornase alfa) (Roche),

The following reaction mixture was pipetted into a 15 ml Falcon tube:
100 μg linearized protein-free DNA,
400 μl 5× buffer (Tris-HCl pH 7.5, MgCl₂, spermidine, DTT, inorganic pyrophosphatase 25 U),
20 μl ribonuclease inhibitor (recombinant, 40 U/μl);
80 μl rNTP-mix (ATP, CTP, UTP 100 mM), 29 μl GTP (100 mM);
116 μl cap analogue (100 mM);
50 μl T7 RNA polymerase (200 U/μl);
1,045 μl RNase-free water.

The total volume was 2 ml and was incubated at 37 °C. for 2 hours in a heating block. Thereafter, 300 μl DNase: Pulmozyme™ (1 U/μl) were added and the mixture was incubated at 37° C. for a further 30 minutes. The DNA template was enigmatically degraded by this procedure.

5. Purification of the mRNAs

### 5.1. LiCl Precipitation (Lithium Chloride/Ethanol Precipitation)

Based on 20-40 μg RNA, this was carried out as follows:

### LiCl Precipitation 25 μl LiCl Solution [8 M]

30 μl WFI (water for injection) were added to the transcription batch (20 μl) and the components were mixed carefully. 25 μl LiCl solution were added to the reaction vessel and the solutions were vortexed for at least 10 seconds. The batch was incubated at −20 °C. for at least 1 hour. The closed vessel was then centrifuged at 4,000 rpm for 30 minutes at 4° C. The supernatant was discarded.

### Washing

5 μl 75% ethanol were added to each pellet (under a safety workbench). The closed vessels were centrifuged at 4,000 rpm for 20 minutes at 4° C. The supernatant was discarded (under a safety workbench) and centrifugation was carried cut again at 4,000 rpm for 2 minutes at 4 °C. The supernatant was carefully removed with a pipette Sunder a safety workbench). Thereafter, the pellet was dried for approx. 1 hour Sunder a safety workbench).

### Resuspension

In each case 10 μl WFI were added to the thoroughly dried pellets (under a safety workbench). The particular pellet was then dissolved in a shaking apparatus overnight at 4 °C.

### 5.2. Final Purification

The final purification was carried out by phenol/chloroform extraction. However, it can likewise be carried out by means of anion exchange chromatography (e.g. MEGAclear™ from Ambion or Rneasy from Qiagen). After this purification of the mRNA, the RNA was precipitated against isopropanol and NaCl (1 M Had 1; 10, isopropanol 1:1), vortexed, and centrifuged at 4,000 rpm for 30 min at 4° C., and the pellet was washed with 75% ethanol. The RNA purified by means of phenol/chloroform extraction was dissolved in RNase-free water and incubated at 4 °C. for at least 12 hours. The concentration of each mRNR was measured at OD₂₆₀ absorption. (The chloroform/phenol extraction was carried out in accordance with Sambrook at, Fritsch E. F., and Maniatis T., in Molecular Cloning; A Laboratory Manual, Cold Spring Harbor Laboratory Press, N.Y., vol. 1,2,3 (1389)).

### Example 2

### Stabilising of the mRNA

An example of an embodiment of the stabilised mRNA according to the invention relates to a β-globin UTR-stabilized mRNA. An mRNA stabilised in this manner had the following structure; cap-β-globin UTR (80 bases)-β-galactosidase coding sequence-β-globin 3′-UTR (approx. 180 bases)-poly A tail (A₃₀C₃₀). Instead of the β-galactosidase coding sequence, constructs which had a sequence which codes for an antigen from a pathogen or tumour already described above were likewise produced.

As a further example of an embodiment of the stabilised mRNA according to the invention, the nucleic acid sequence of the coding region of the mRNA was optimised in respect of its G/C content. To determine the sequence of a modified mRNA according to the invention, the computer program described in WO 02/098443 was used, which, with the aid of the genetic code or the degenerative nature thereof, modifies the nucleotide sequence of any desired mRNA such that a maximum G/C content results, in combination with the use of codons which code for tRNAs occurring as frequently as possible in the cell, the amino acid sequence coded by the modified mRNA preferably being identical to the non-modified sequence. Alternatively, it is also possible to modify only the G/C content or only the codon usage compared with the original sequence. The source code in Visual Basic 6.0 (development environment used; Microsoft Visual Studio Enterprise 6.0 with Servicepack 3) is likewise described in WO 02/038443, the disclosure of which is subject matter of the present invention.

### Example 3

### Cell Culture

P815 cells were supplemented with 10% heat-inactivated foetal calf serum (PAN systems, Germany), 2 mM L-glutamine, 100 U/ml penicillin and 100 μg/ml streptomycin and cultured in an RPMI 1640 (Bio-Whittaker, Verviers, Belgium). The CTL culture was carried out in RPMI 1640 medium, supplemented with 10% FCS, 2 mM L-glutamine, 100 U/ml penicillin, 100 μg/ml streptomycin, 50 μM β-mercaptoethanol, 50 μg/ml gentamycin, 1×MEM non-essential amino acids and 1 mM sodium pyruvate. The CTLs were restimulated for one week with 1 μg/ml β-galactosidase (Sigma, Taufkirchen, Germany). On day 4, the supernatants were carefully collected and replaced by fresh medium containing 10 U/ml rIL2 (final concentration).

In parallel experimental set-ups, the restimulation was carried out with in each case 1.3 μg/ml survivin, 1 μg MAGE-3 and 0.8 μg Muc-1. All the other conditions in these experimental set-ups were identical to the conditions described above,

### Example 4

### Immunization of Mice

Female BALB/c AnNCrlBR (H-2d) mice 6 to 12 weeks old were obtained from Charles River (Suizfeid, Germany). Approval for the genetic (DNA and mRNA) vaccination of the mice was granted by the Committee for Animal Ethics in Tubingen (number IM/200). The BALE mice were anaesthetized with 20 mg pentobarbital intraperitoneally. The mice were then injected intradermally in both ear pinnae with 25 μg β-globin UTR-stabilized mRNA coding for β-galactosidase, which was diluted with injection buffer (150 mM NaCl, 10 mM HEPES). 5·10³ units (1 μg) of GM-CSF (Peprotech, Inc., Rocky Hill, N.Y., USA), diluted with 25 μl PBS, were subsequently injected. This corresponded to a total amount of 2 μg (approx, 10⁴ units), which was injected only once. Such a dosage lies in the lowest, range of the dosages normally chosen in mice (26). Two weeks after the first injection, the mice were treated under the same conditions (as with the first injection).

In parallel experimental set-ups I, II+III, which were carried out under the same conditions described above, mice were injected with, instead of 25 μg β-globin UTR-stabilized mRNA which coded for β-galactosidase and 1 μg GM-CSF, in
Experimental set-up I: 30 μg β-globin UTR-stabilised mRNA coding for survivin and 1.2 μg IL-2, in
Experimental set-up II: 23 μg β-globin UTR-stabilised mRNA coding for MAGE-3 and 2 μg IL-12, and in
Experimental set-up III: 18 μg β-globin UTR-stabilised mRNA coding for Muc-1 and 1 μg IFN-α.

GM-CSF (total amount of 2 μg of recombinant protein: approx, 10⁴ U (units)) were injected once, either 24 hours or 2 hours before injection of the mRNA or 24 hours after injection of the mRNA (corresponds to groups GM-CSF T1, GM-CSF T-0 and GM-CSF T+1). The amount of β-galactosidase-specific IgG1 or IgG2a antibodies contained in the blood of the injected mice was determined by ELISA (1:10 serum dilution). The background, which was chiefly obtained by the serum of buffer-injected mice at the same dilution, was subtracted.

### Example 5

### Chromium Release Assay

Splenocytes were stimulated in vitro with purified β-galactosidase (1 mg/ml) and the CTL activity was determined after 6 days using a standard ⁵¹Cr release assay (as described, for example, by Rammensee et al. (1939), Immunogenetics 30: 296-302). The death rate of the cells was determined with the aid of the amount of ⁵¹Cr released into the medium (A) compared with the amount of spontaneous ⁵¹Cr release of the target cells (B) and the total content of ⁵¹Cr of target cells lysed with 1% Triton-X-100 (C) by means of the formula

% cell lysis=(A−B)+(C−B)×100

Stimulation of the splenocytes with survivin. MAGE-3 and Muc-1 (concentration in each case 1 mg/ml) was carried out in parallel experimental set-ups. All the other conditions in these experimental set-ups were identical to the conditions described above.

### Example 6

### ELISA

MaxiSorb plates from Nalgene Nunc International (Nalge, Denmark) were coated overnight at 4° C. with 100 μl β-galactosidase at a concentration of 100 μg/ml (antibody ELISA) or with 50 μl of anti-mouse anti-IFN-γ or -IL-4 (cytokine ELISA) capture antibodies (Becton Dickinson, Heidelberg, Germany) at a concentration of 1 μg/ml in coating Puffer (0.02% NaN₃, 15 mM Na₂CO₃, 15 mM NaHCO₃, pH 9.6). The plates were then saturated for 2 hours at 37° C. with 200 μl of blocking buffer (PBS-0.05% Two en 20-1% BSA). They were subsequently incubated at 37° C. for 4 to 5 days with sera (antibody ELISA) at 1:10, 1:30 and 1:90 dilutions in washing buffer or 100 μl of the call culture supernatant (cytokine ELISA). 100 μl of 1:1,000 dilutions of goat anti-mouse IgG1 or IgG2a antibodies (antibody ELISA) from Caltag (Burlington, Calif., GSA; or 100 μl/well of biotinylated anti-mouse anti-IFN-γ or -IL-4 (cytokine ELISA) detection antibodies (Becton Dickinson, Heidelberg, Germany) at a concentration of 0.5 μg/ml in blocking buffer were then added and the plates were Incubated at room temperature for 1 hour.

For the cytokine ELISA, after 3 washing steps with washing buffer, 100 μl of a 1:1,000 dilution of streptavidin-HRP (BD Biosciences, Heidelberg, Germany) were added per well. After 30 minutes at room temperature, 100 μl ABTS (2,2′-azino-bis-(3-ethylbenzothiazoline-6-sulfonic acid) concentrate at a concentration of 300 mg/l in 0.1 M citric acid, pH 4.35) were added per well. After a further 15 to 30 min at room temperature, the extinction at OD₄₆₅ was measured with a Sunrise ELISA reader from Tecan (Crailsheim, Germany). The amounts of the cytokines were calculated with the aid of a standard curve plotted by titration of certain amounts of recombinant cytokines (BD Pharmingen, Heidelberg, Germany).

In parallel experimental set-ups, the MaxiSorb plates were coated with survivin, MAGE-3 and Muc-1 (in each case 100 μl). All the other conditions in these parallel experimental set-ups were identical to the conditions described above.

### Example 7

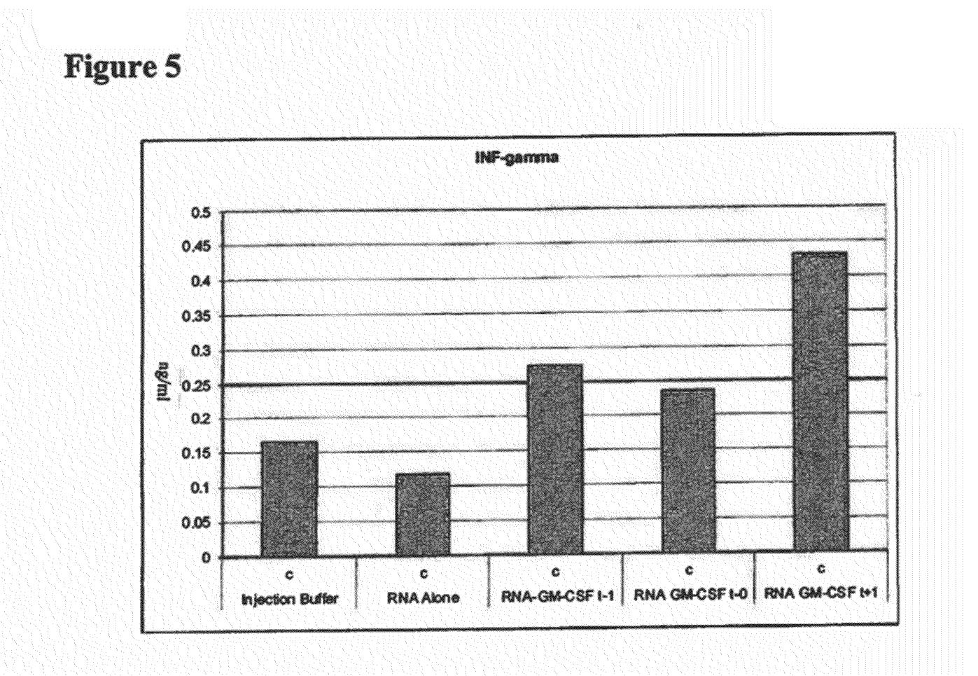

### Immunization of Mice with GM-CSF RNA (cf. FIG. 5)

Female BALB/c AnNCr1BR(H-2d) mice 6 to 12 weeks old (Charles River, Suizfeld, Germany) BALE mice were anaesthetized with 20 mg pentobarbital intraperitoneally analogously to Example 4 (see above). The mice were then injected intradermally in both ear pinnae with 25 μg of β-globin UTR-stabilized mRNA coding for β-galactosidase, which was diluted with injection buffer (150 mM NaCl, 10 mM HEPES). 50 μg GM-CSF RNA were subsequently injected once into the ear pinnae. Two weeks after the first injection, the mice were treated under the same conditions (as with the first injection).

In parallel experimental set-ups I, II, III, IV and V, which were carried out under the same conditions described above, mice were, in
Experimental set-up I: injected only with injection buffer (control);
Experimental set-up II: injected with 50 μg GM-CSF RNA alone (control);
Experimental set-up III: injected with 25 μg β-globin UTR-stabilised mRNA which coded for β-galactosidase, and 50 μg GM-CSF RNA, the GM-CSF RNA being administered 24 hours before the β-globin UTR-stabilized mRNA coding for β-galactosidase (corresponding to t−1);
Experimental set-up-IV: injected with 25 μg β-globin UTR-stabilized mRNA which coded for β-galactosidase, and 50 μg GM-CSF RNA, the GM-CSF RNA being administered 2 hours before the β-globin UTR-stabilised mRNA coding for β-galactosidase (corresponding to t−0);
Experimental set-up V: injected with 25 μg β-globin UTR-stabilized mRNA which coded for β-galactosidase, and 50 μg GM-CSF RNA, the GM-CSF BRA being administered 24 hours after the β-globin UTR-stabilized mRNA coding for β-galactosidase (corresponding to t+1).

Mazi Sorb plates from Nalgene Nunc International (Nalge Denmark) were plated out overnight at 4° C. with 50 ml of an anti-moose anti-interferon-γ (IFN-γ) antibody with 1 mg/ml in a coating buffer (0.02% NaN₃, 15 mM Na₂CO₃, 15 mM NaHCO₃, pH 6.6). The plates were then saturated with 200 ml of the blocking buffer (PBS-0.05% Two on 20-1% BSA) for 2 hours at 37° C. and then incubated at 37° C. for 4-5 h with 100 ml of the cell culture supernatant (cytokine ELISA), 100 μl of 1:1,000 dilutions of 100 μl per well of the biotinylated anti-mouse anti-IFN-γ detection antibody (Becton Dickinson) were added at 0.5 mg/ml in a blocking buffer and incubation was carried out at room temperature for one hour. After 3 washing steps with washing buffer, 100 ml of a 1 to 1,000 dilution of streptavidin-HRP (horseradish peroxidase, BD Biosciences, Heidelberg, Germany) were added per well. After 30 minutes at room temperature, 100 ml per well of ABTS (300 mg/l 2,2-azino-bis-(3-ethylbenzothiazoline-6 sulfonic acid) in 0.1 M citrate, pH 4.35) substrate were added. After 15 to 30 minutes at room temperature, the extinction at OD405 was measured with a Sunrise ELISA reading apparatus from Tecan (Crailsheim, Germany) and the amounts of the cytokine were calculated from a standard curve which was obtained by titration with certain amounts of recombinant cytokines (BD Pharmingen, Heidelberg, Germany). It can be clearly seen that the immunostimulation is significantly increased by administration of GM-CSF mRNA before, at about the same time as and after injection of β-galactosidase mRNA.

### BIBLIOGRAPHY

1. Tang, D. C., DeVit, M. & Johnston, S. A. Genetic immunisation is a simple method for eliciting an immune response. Nature 356, 152-154 (1932).
2. Ulmer, J. B. et al. Heterologous protection against influenza by injection of DNA encoding a viral protein. Science 259, 1745-1749 (1993).
3. Wang, B. et al. Gene inoculation generates immune responses against human immunodeficiency virus type 1. Proc Natl Acad Sci U S A 90, 4156-4160 (1993).
4. Robinson, H. L., Hunt, L. A. & Webster, R. G. Protection against a lethal influents virus challenge by immunisation with a haemagglutinin-expressing plasmid DNA. Vaccine 11, 957-960 (1993.
5. Fynan, E. F. et al. DNA vaccines: protective immunizations by parenteral, mucosal, and gene-gun immunizations. Proc Natl Acad Sci U S A 90, 11478-11482 (1993).
6. Ulmer, J. B. An update on the state of the art of DNA vaccines. Curr. Opin. Drug Discov. Devel. 4, 192-197 (2001).
7. Donnelly, J., Berry, K. & Ulmer, J. B. Technical and regulatory hurdles for DNA vaccines. Int J Parasitol. 33, 457-467 (2003.
8. Klinman, D. M. et al. DNA vaccines: safety and efficacy issues. Springer Semin. Immunopathol. 19, 245-256 (1997).
9. Gilkeson, G. S., Pippen, A. M. & Pisetsky, D. S. Induction of cross-reactive anti-dsDNA antibodies in preautoimmune NZB/NZW mice by immunization with bacterial DNA. J Clin Invest 95, 1398-1402 (1995).
10. Saenz-Badillos, J., Amin, S. P. & Granstein, R. D. RNA as a tumour vaccine: a review of the literature, Exp Dermatol 10, 143-154 (2001).
11. Sullenger, B. A. & Gilboa, E, Emerging clinical applications of RNA, Nature 418, 252-258 (2002).
12. Nair, S. K. et al. Induction of primary carcinoembryonic antigen (CEA)-specific cytotoxic T lymphocytes in vitro using human dendritic cells transfected with RNA. Nat Biotechnol 16, 364-369 (1998).
13. Ying, H. et al. Cancer therapy using a self-replicating RNA vaccine. Nat. Med. 5, 823-827 (1999).
14. Schirmacher, V. et al. Infra-pinna anti-tumour vaccination with self-replicating infectious RNA or with DNA encoding a model tumour antigen and a cytokine. Gene Ther. 7, 1137-1147 (2000).
15. Martinon, F. et al. Induction of virus-specific cytotoxic T lymphocytes in vivo by liposome-entrapped mRNA. Eur J Immunol 23, 1719-1722 (1393).
16. Vassilev, V. B., Gil, L. H. & Donis, R. O. Microparticle-mediated RNA immunisation against bovine viral diarrhea virus. Vaccine 19, 2012-2019 (2001).
17. Hoerr, I., Obst, R., Rammensee, H. G. & Jung, G. In vivo application of RNA leads to induction of specific cytotoxic T lymphocytes and antibodies. Eur. J Immunol. 30, 1-7 (2000).
18. Granstein, R. D., Ding, W. & Ozawa, H. Induction of anti-tumour immunity with epidermal cells pulsed with tumour-derived RNA or intradermal administration of RNA. J Invest Dermatol 114, 632-636 (2000).
19. Iwasaki, A., Stiernholm, B. J., Chan, A. K., Berinstein, N. L. & Barber, B. H, Enhanced CTL responses mediated by plasmid DNA immunogens encoding costimulatory molecules and cytokines, J Immunol 158, 4591-4601 (1997).
20. Warren, T. L. & Weiner, G. J. Uses of granulocyte-macrophage colony-stimulating factor in vaccine development. Curr. Opin. Hematol. 7, 168-173 (20005.
21. Scheel, B. et al, Immunostimulating capacities of stabilised RNA molecules. Eur. J. Immunol. 34, 537-547 (2004).
22. Diebold, S. S., Kaisho, T., Hemmi, H., Akira, S. & Reis E Sousa. Innate antiviral responses by means of TLR7-mediated recognition of single-stranded RNA, Science 303, 1529-1531 (2004).
23. Heil, F. et al. Species-specific recognition of single-stranded RNA via toll-like receptor 7 and 8. Science 303, 1526-1529 (2004).
24. Kwissa, M., Kroger, A., Hauser, H., Reimann, J. & Schirmbeck, R. Cytokine-facilitated priming of CD8(+) T cell responses by DNA vaccination. J Mol. Med. 81, 91-101-2003).
25. Cho, J. H., Lee, S. W. & Sung, Y. C. Enhanced cellular immunity to hepatitis C virus nonstructural proteins by codelivery of granulocyte macrophage-colony stimulating factor gene in intramuscular ilea immunization. Vaccine 17, 1136-1144 (1999).
26. Weber, J. et al. Granulocyte-macrophage-colony-stimulating factor added to a multipeptide vaccine for resected Stage II melanoma. Cancer 97, 186-200 (2003).
27. Kusakabe, K. et al. The timing of GM-CSF expression plasmid administration influences the Th1/Th2 response induced by an HIV-1-specific DNA vaccine. J Immunol 164, 3102-3111 (2000).

1-20. (canceled)

21. A method for stimulating an immune response in a mammal comprising the following steps:
(a) administering to the mammal at least one antigen in RNA which codes for at least one antigen of a pathogen or codes for at least one tumour antigen; and
(b) administering to the mammal at least one cytokine mRNA.

22. The method of claim 21, wherein the at least one antigen mRNA from step (a) contains a region which codes for at least one antigen from a tumour selected from the group consisting of 707-AP, AFP, ART-4, BAGE, β-catenine/m, Bcr-abl, CAMEL, CAP-1, CASP-8, CDC27/m, CDK4/m, CEA, CMV pp65, CT, Cyp-B, DAM, EGFRI, ELF2M, ETV6-AML1, G250, GAGE, GnT-V, Gp100, HAGE, HBS, HER-2/neu, HLA-A*0201-R170I, HPV-E7, HSP70-2M, HAST-2, hTERT (or hTRT), influenza matrix protein, in particular influenza A matrix M1 protein or influenza B matrix M1 protein, iCE, KIAA0205, LAGE, e.g. LAGE-1, LDLR/FUT, MAGE, e.g. MAGE-A, MAGE-B, MAGE-C, MAGE-A1, MAGE-2, MAGE-3, MAGE-6, MAGE-10, MART-1/melan-A, MC1R, myosine/m, MUC1, MUM-1, -2, -3, NA88-A, NY-ESO-1, p190 minor bcr-abl Pml/RARα, PRAME, proteinase 3, PSA, PSM, PTPRZ1, RAGE, RU1 or RU2, SAGE, SART-1 or SART-3, SEC61G, SOX9, SPC1, SSX, surviving, TEL/AML1, TERT, TNC, TPI/m, TRP-1, TRP-2, TRP-2/INT2, tyrosinase and WT1.

23. The method of claim 21, further comprising administering an RNase inhibitor in step (a).

24. The method of claim 23, wherein the RNase inhibitor is selected from the group consisting of RNasin and aurintricarboxylic acid.

25. The method of claim 21, wherein the at least one cytokine is selected from the group consisting of IL-1 (α/β), IL-2, IL-3, IL-4, IL-5, IL-6, IL-7, IL-8, IL-9, IL-10, IL-12, IL-13, IL-15, IL-18, IL-21, IL-22, IL-23, IFN-α, IFN-β, IFN-γ, LT-α, MCAF, RANTES, TGFα, TGFβ1, TGFβ2, TNFα, TNFβ, G-CSF, GM-CSF and M-CSF.

26. A product comprising at least one antigen mRNA containing a region which codes for at least one antigen of a pathogen or at least one tumour antigen, and at least one component from at least one of the following categories selected from the group consisting of a cytokine, a CpG DNA, a cytokine mRNA, an adjuvo-viral mRNA, and an adjuvant RNA, as a combination preparation for simultaneous, separate or time-staggered use in the treatment and/or prophylaxis of tumour diseases, allergies, autoimmune diseases, viral Infections, and bacterial infections.

27. The method according to claim 21, wherein step b. is carried out over: 1 minute to 48 hours, 20 minutes to 26 hours, 30 minutes to 24 hours; 10 hours to 30 hours, 12 hours to 28 hours, or 20 hours to 26 hours.

28. The method according to claim 21, wherein at least one mRNA from step (a.) and/or from step (b.) is in the form of naked or complexed mRNA.

29. The method according to claim 21, wherein at least one mRNA from step (a.) and/or from step (b.) is in the form of globin UTR (untranslated regions)—stabilized mRNA.

30. The method according to claim 29, wherein stabilized mRNA is β-globin UTR-stabilized mRNA.

31. The method according to claim 21, wherein at least one mRNA from step (a.) and/or from step (b.) is in the form of modified mRNA.

32. The method according to claim 31, wherein the modified mRNA is stabilized mRNA.

33. The method according to claim 21, wherein the G/C content of the coding region of the modified mRNA from step (a.) and/or from step (b.) is increased compare with the G/C content of the coding region of the wild-type RNA, and wherein the coded amino acid sequence of the modified mRNA is not modified compare with the coded amino acid sequence of the wild-type mRNA.

34. The method according to claim 21, wherein the A/U content in the environment of the ribosome binding site of the modified mRNA from step (a.) and/or from step (b.) is increased compared with the A/U content in the environment of the ribosome binding site of the wild-type mRNA.

35. The method according to claim 21, wherein the coding region and/or the 5′ and/or 3′ untranslated region of the modified mRNA from step (a.) and/or from step (b.) is modified compared with the wild-type mRNA such that it contains no destabilizing sequence elements, wherein the coded amino acid sequence of the modified mRNA is not modified compared with the wild-type mRNA.

36. The method according to claim 21, wherein the modified mRNA from step (a.) and/or from step (b.) comprises a 5′ cap structure and/or a poly(A) of at least 25 nucleotides, and/or at least one IRES and/or at least one 55 and/or 3′ stabilizing sequences.

37. The method according to claim 21, wherein the modified mRNA from step (a.) and/or from step (b.) or the adjuvant RNA from step (b.) contains at least one analogue of naturally occurring nucleotides.

38. The method according to claim 21, wherein the modified mRNA from step (a.) and/or from step (b.) or the adjuvant RNA from step (b.) is completed or condensed with at least one cationic or polycationic agent.

39. The method according to claim 38, wherein the cationic or polycationic agent is selected from the group consisting of protamine, poly-L-lysine, poly-L-arginine, and histones.

40. The method of claim 21 for the treatment of tumor diseases, allergies, autoimmune diseases, multiple sclerosis, and protozoological, viral, and/or bacterial infections.


SEQUENCE LISTING (5 sequences)

SEQ ID NO: 1 - RNA, 13 nt, Artificial Sequence
  Description of Artificial Sequence Synthetic Kozak sequence (see description p. 30)
gccgccaccaugg

SEQ ID NO: 2 - RNA, 15 nt, Artificial Sequence
  Description of Artificial Sequence Synthetic RNA stabilizing sequence (see description p. 33)
nccancccnnucncc

SEQ ID NO: 3 - DNA, 45 bp, Xenopus sp.
  misc_feature (1)..(45): Xenopus beta-globin 5′-untranslated region (see description p. 47, Diagram 1)
gcttgttctttttgcagaagctcagaataaacgctcaactttggc

SEQ ID NO: 4 - DNA, 157 bp, Xenopus sp.
  misc_feature (1)..(157): Xenopus beta-globin 3′-untranslated region (see description p. 47, Diagram 1)
gactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattcta

SEQ ID NO: 5 - DNA, 48 bp, Homo sapiens
  misc_feature (1)..(48): human alpha-globin untranslated region (see description p. 47, Diagram 1)
ctagtgactgatagcccgctgggcctcccaacgggccctcctcccctc